(19) United States
(12) Patent Application Publication
(10) Pub. No.: US 2008/0275298 A1
(43) Pub. Date: Nov. 6, 2008

(54) Dual View Endoscope

(75) Inventor: Nitesh Ratnakar, Elkins, WV (US)

Correspondence Address: NITESH RATNAKAR, ROUTE 3,, BOX 179-A, ELKINS, WV, 26241, US

(73) Assignee: NOVATION SCIENCE, LLC, Elkins, WV (US)

(21) Appl. No.: 12/172,237

(22) Filed: Jul. 13, 2008

Related U.S. Application Data
(63) Continuation-in-part of application No. 10/711,859, filed on Oct. 11, 2004.
Application No. 10/711,859 is a continuation-in-part of application No. 10/908,300, filed on May 6, 2005.
Application No. 10/908,300 is a continuation-in-part of application No. 11/778,987, filed on Jul. 17, 2007.

(51) Int. Cl. A61B 1/055

(52) U.S. Cl. 600/109; 600/113

(57) ABSTRACT

The present invention relates to endoscope system having means to provide simultaneous forward and rear views of a hollow organ. It comprises of first image assembly comprising of forward image lens and forward illumination source disposed at distal end of the endoscope and; second image assembly comprising of rear image lens and rear illumination source disposed on rear view module operatively engaged with the endoscope. Upon deployment of rear view module, rear image lens and rear illumination source face backwards with respect to the endoscope; and in some embodiments of the invention; face towards forward image lens and illumination source disposed at the distal end of the endoscope. According one aspect of the invention, means is provided to prevent interference between the forward image lens of the endoscope and rear illumination source of the rear view module; and between rear image lens of the rear view module and forward illumination source of the endoscope.

### FIELD OF INVENTION:

The present invention relates to endoscopes, more specifically to means and methods to improve image quality of endoscope having means to provide simultaneous forward and rear views of hollow organ.

### BACKGROUND AND PRIOR ART

Endoscopes are used to perform a variety of surgical procedures. Endoscopes currently in use only provide forward view of hollow organ. Currently, rear view of hollow organ is obtained by retro-flexing the endoscope by 180 degrees; as shown in FIGS. 3, 4A & 4B. However rear-flexion is usually not possible in most hollow organs of the body and can lead to complications such as perforation. Moreover, even with retro-flexion, present endoscopes only enable one view at a time; as a result of which operator has to switch between forward and retroflexed positions to enable complete visualization of hollow organ. This is not only time consuming but also increases the rates of procedural complications.

In our US publications 2005/0038317 and 2006/025994; included as reference herein, endoscope with means to obtain simultaneous forward and rear view of hollow organ is disclosed. In summary, various embodiments of the dual view endoscope in US publication 2005/0038317; and included in entirety as reference herein; contain forward view and rear view modules; each having independent image lens and illumination source. Means is provided to move the forward and rear view modules independently of each other so as to enable the operator to position the forward view module in forward viewing position and rear view module in rear viewing positioning. However, in this position, the rear illumination source faces the forward image lens and the forward illumination source faces the rear image lens which leads to interference; and consequently impairs the quality of both forward and rear images. Various preferred embodiments of dual view endoscope are shown in FIGS. 5-31 and included as reference herein.

### SUMMARY OF THE INVENTION:

In light of the significant limitations discussed above, there is a need for means to prevent interference between the forward and rear facing imaging systems in a dual view endoscope and improve quality of both forward and rear images. Additional features and advantages of the present invention will be set forth in the description and drawings which follow or may be learned by practice of the invention.

### BRIEF DESCRIPTION OF THE DRAWINGS:

FIG. 1 shows a view of a conventional endoscope.

FIG. 2 shows a side view of the distal end, bending section and insertion tube of a conventional endoscope.

FIG. 3 is a side view of a conventional endoscope displaying the field of vision of a conventional endoscope.

FIG. 4A shows a conventional endoscope inside a colon. It shows mucosal folds of the colon and illustrates that visualization of the area behind mucosal folds is obstructed by the front of the mucosal folds during examination with a conventional endoscope.

FIG. 4B shows a conventional endoscope inside a colon in a retroflexed position. It displays how rear flexion enables visualization of area behind a mucosal fold.

FIG. 5 shows side view of an endoscope with the ‘rear view module’ according to a first embodiment of the present invention.

FIG. 6 shows side view of the endoscope in FIG. 5 wherein the ‘rear view module’ is deployed for rear view.

FIG. 7 shows side view of an endoscope with the ‘rear view module’ according to a second embodiment of the present invention.

FIG. 8 shows side view of the endoscope in FIG. 7 wherein the ‘rear view module’ is deployed for rear view.

FIG. 9 shows side view of an endoscope with a ‘rear view module’ according to a third embodiment of the present invention.

FIG. 10 is a side view of the endoscope in FIG. 9 wherein the ‘rear view module’ is deployed for rear view.

FIG. 11 shows side view of an endoscope with a ‘rear view module’ according to a fourth embodiment of the present invention.

FIG. 12 shows side view of the endoscope in FIG. 11 wherein the ‘rear view module’ is deployed for rear view.

FIG. 13 shows side view of an endoscope with a ‘rear view module’ according to a fifth embodiment of the present invention.

FIGS. 14-16 show a side view of the endoscope in FIG. 13 wherein the ‘rear view module’ is deployed for rear view.

FIG. 17 shows side view of a ‘rear view module’ according to a sixth embodiment of the present invention.

FIGS. 18 & 19 shows side view of the endoscope in FIG. 17 wherein the ‘rear view module’ is deployed for rear view.

FIG. 20 shows side view of an endoscope with a ‘rear view module’ according to a seventh embodiment of the present invention.

FIG. 21 is a side view of the endoscope in FIG. 20 wherein the ‘rear view module’ is deployed for rear view.

FIG. 22 shows side view of an endoscope with a ‘rear view module’ according to an eighth embodiment of the present invention.

FIG. 23 is a side view of the endoscope in FIG. 22 wherein the ‘rear view module’ is deployed for rear view.

FIG. 24 shows side view of an endoscope with a ‘rear view module’ according to a ninth embodiment of the present invention.

FIG. 25 is a side view of the endoscope in FIG. 24 wherein the ‘rear view module’ is deployed for rear view.

FIG. 26 shows side view of an endoscope with a ‘rear view module’ according to a tenth embodiment of the present invention.

FIG. 27 is a side view of the endoscope in FIG. 26 wherein the ‘rear view module’ is deployed for rear view.

FIG. 28 shows side view of an endoscope with a ‘rear view module’ according to an eleventh embodiment of the present invention.

FIG. 29 is a side view of the endoscope in FIG. 28 wherein the ‘rear view module’ is deployed for rear view.

FIG. 30 shows side view of an endoscope with a ‘rear view module’ according to a twelfth embodiment of the present invention.

FIG. 31 is a side view of the endoscope in FIG. 30 wherein the ‘rear view module’ is deployed for rear view.

FIGS. 32A-32I show various arrangements of polarizing light filters disposed in relation to the endoscope and rear view module and having means to prevent light interference.

FIG. 33A shows forward and rear illumination source operatively connected to light intensity control system having to means to automatically adjust light intensity of the forward and rear illumination source to achieve optimal light intensity in common view field.

FIG. 33B shows forward and rear image lens operatively connected to image light exposure adjustment application having to means to automatically adjust light exposure of images captured by forward and rear image lens to achieve optimal image quality.

FIG. 34 shows forward and rear illumination sources connected to alternating switch having means to supply power alternatively to forward illumination and rear illumination source.

FIG. 35A shows forward and rear illumination sources, each having shutter to block passage of light, shutter comprising of rotating disc. It further shows rotating discs connected to motor having means to rotate the rotating discs, the rotation of the forward and rear rotating discs synchronized such that when forward rotating disc allows passage of light from forward illumination source, the rear rotating disc blocks light passage from rear illumination source.

FIG. 35B shows forward and rear illumination sources, each having shutter to block passage of light, shutter comprising of opaque screen. It further shows opaque screens connected to motor having means to move the opaque screens, the movement of the forward and rear opaque screens synchronized such that when forward opaque screen allows passage of light from forward illumination source, the rear opaque screen blocks light passage from rear illumination source.

FIG. 36 shows forward and rear illumination sources remote from the distal tip of the endoscope and rear view module respectively, light guide having means to transmit light from forward and rear illumination sources to the distal tip of the endoscope and rear view module respectively; remote forward and rear illumination sources having shutter to block passage of light, shutter comprising of rotating disc. It further shows rotating discs connected to motor having means to rotate the rotating discs, the rotation of the forward and rear rotating discs synchronized such that when forward rotating disc allows passage of light from remote forward illumination source, the rear rotating disc blocks light passage from remote rear illumination source.

FIG. 37 shows rear view module extending outwards from the distal tip of the endoscope and; rear light source disposed on the endoscope proximal the position of the forward illumination source.

FIG. 38 shows rear view module disposed inside hollow channel of the endoscope and extending outwards from the distal tip of the endoscope; distal end of the rear view module having inflatable balloon operatively connected to air pump; inflatable balloon inflated inside the distal hollow channel of the endoscope securing the position of the rear view module with respect to the endoscope.

FIG. 39 shows rear view module disposed inside hollow channel of the endoscope and extending outwards from the distal tip of the endoscope; distal end of the rear view module having resistance beads in contact with inner surface of the hollow channel of the endoscope; securing the position of the rear view module with respect to the endoscope.

FIG. 40 shows rear view module disposed inside hollow channel of the endoscope and extending outwards from the distal tip of the endoscope; distal end of the hollow channel having elevator operatively connected to an actuator, elevator having means to engage rear view module disposed inside the hollow channel; securing the position of the rear view module with respect to the endoscope.

FIGS. 41A & 41B show rear view module disposed inside hollow channel of the endoscope and extending outwards from the distal tip of the endoscope; distal end of the rear view module having magnet means; distal end of the hollow channel having metal rings; magnet means engaged with metal rings inside the distal hollow channel of the endoscope securing the position of the rear view module with respect to the endoscope.

FIGS. 42A & 42B show rear view module disposed inside hollow channel of the endoscope and extending outwards from the distal tip of the endoscope; distal end of the rear view module having locking contraption; distal end of the hollow channel having mating contraption corresponding to locking contraption and; engagement of the locking contraption with mating contraption securing the position of the rear view module with respect to the endoscope.

### DETAILED DESCRIPTION OF THE DRAWINGS

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out one or several purposes of the present invention. It is important, therefore, that the claims be regarded as including such equivalent constructions insofar as they do not depart from the spirit and scope of the present invention.

Reference will now be made in detail to the preferred embodiments of the invention, examples of which are illustrated in the accompanying drawings. Wherever possible, the same reference numbers will be used throughout the drawings to refer to the same or like parts. The following general description applies to preferred embodiments of the present invention.

The dual view endoscope comprises of a rear view module. Rear view module is a solid structure that can be rectangular, square, tubular, discoid or of any other shape. It is attached to a conventional endoscope by a suitable mechanical articulation such as ball socket joint, hinge joint, bi planar rolling joint etc. The rear view module consists of a rear image lens to obtain a rear view. The rear image lens is attached to an image processor by an electric cable. This cable transmits the image obtained by the rear image lens to the image processor. After being processed, the image is then viewed on a computer monitor or any other display unit. The rear view module also contains a rear illumination bulb. The rear illumination bulb is connected to a power source by an electric cable. The rear illumination bulb illuminates the area under view of the rear image lens. The rear image lens and the rear illumination bulb are typically activated upon deployment of the rear view module. The rear view module is deployed using an actuator. A rear instrument channel is provided in the present invention. It is placed proximal to the rear view module. This channel is connected to the main instrument channel and the passage is controlled by a control valve. Typically, deployment of the rear view module opens the passage to the rear instrument channel. The rear instrument channel is used to pass surgical instruments to do various surgical procedures in areas under view of the rear image lens. It is also used to apply suction in the area under view of the rear image lens. A rear air/water channel is provided. It is placed proximal to the rear view module. The rear air/water channel is connected to the air/water channel of the main endoscope and the passage is controlled by a valve. Typically, deployment of the rear view module opens the passage to the rear air/water channel. The rear air/water channel is used to insufflate air in the direction of view of the rear image lens for better distension and visualization. The air/water channel is also used to squirt water or air at the rear image lens and the rear illumination bulb. This enables cleaning of the rear image lens and the rear illumination bulb while still inside a hollow body organ.

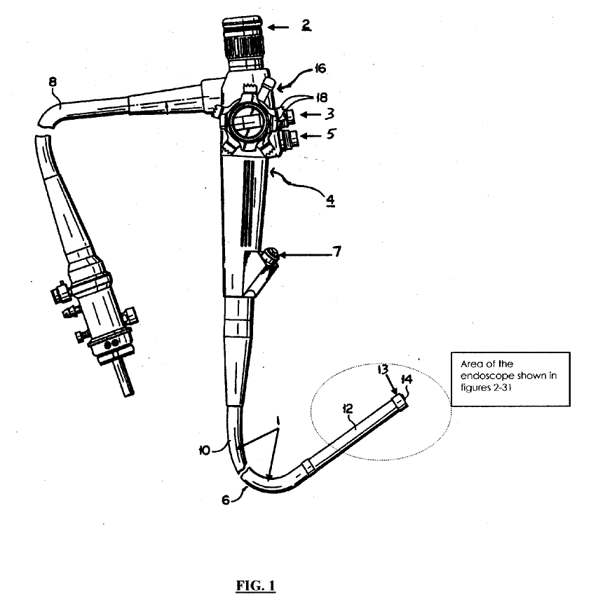

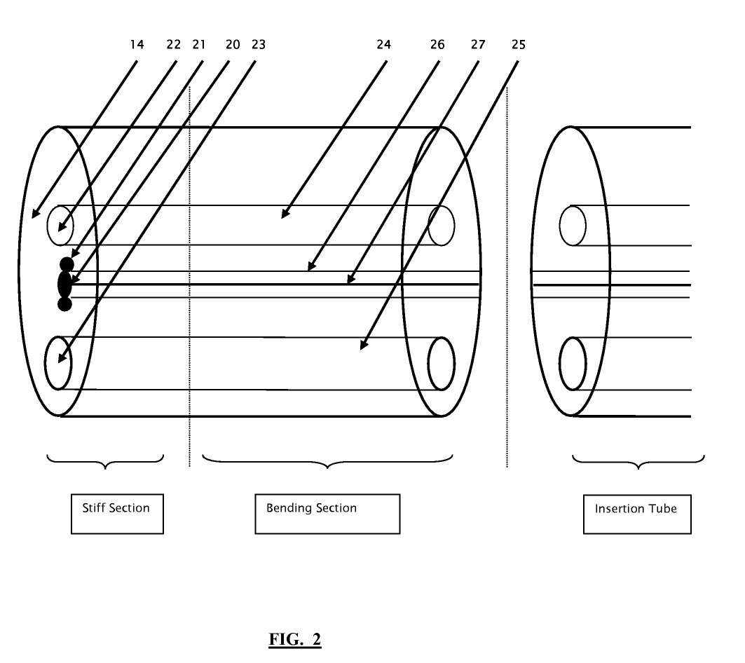

FIGS. 1 and 2 illustrate an embodiment of a conventional endoscope. It has a handle (4) from which extends a flexible shaft (1), which is inserted into a hollow organ to be inspected. The shaft consists of a proximal section (10), insertion tube (6), bending section (12) and a stiff section (13). The shaft terminates in the distal end (14), which typically houses one image lens (20), one to two illumination bulbs (21), air/water nozzle (22) and an instrument channel outlet (23). Light is transmitted from a power source through the shaft via an electric cable (26) to the illumination bulb (21). The illumination bulb illuminates the area to be examined. The image lens (20) captures images of the illuminated area. The image is then transmitted through a fiber optic cable (27) and viewed through an eyepiece (2) attached to the handle of the endoscope. Alternatively, the image is converted to a video signal and is then transmitted to an image processor by an electrical cable. The image is processed and displayed on a display unit like a computer monitor (not shown). The handle (4) of the endoscope has a grip (16) and an extension arm (8) that attaches the endoscope to a power source and an image processor. To enable the endoscope to maneuver through the turns of a hollow organ the shaft is flexible and incorporates a multitude of wires that attach the bending portion (12) with actuators (18). Typically, there are two pairs of such wires passing within the shaft, one pair for flexing the bending portion in one plane and the other pair for flexing it in an orthogonal plane. Tension is applied to these wires using the actuators (18) to move the bending portion (12) in various directions. It is also usual to provide two channels extending between the handle and the distal end of the shaft, an air/water channel (24) and an instrument channel (25). The air/water channel (24) is used to insufflate air in a hollow organ to expand it for proper visualization. The air/water channel is connected proximally to an air/water pump (not shown) and to distally to the air/water nozzle (22). It is controlled by an air/water control valve (5) located on the handle (4). The image lens (20) and the illumination bulb (21) are frequently smeared with blood, stool or other body fluids while in a hollow organ. In such a situation, the air/water channel (24) is used to squirt water or blow air at the image lens (20) and/or illumination bulb (21) in order to clean them while still inside a hollow organ. The instrument channel (25) has an instrument channel inlet (7) proximally and an instrument channel outlet (23) distally. It is used to pass surgical instruments to do various surgical procedures. It is also used to apply suction using the suction control valve (3) located on the handle (4). This suction is useful in removing fluids, air and other materials from within a hollow organ during examination.

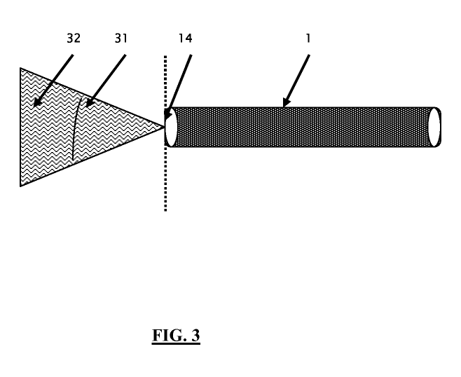

FIG. 3 illustrates the narrow field of vision (31) of about 120 degrees of a conventional endoscope (1). It also shows that conventional endoscopes are only forward viewing (32).

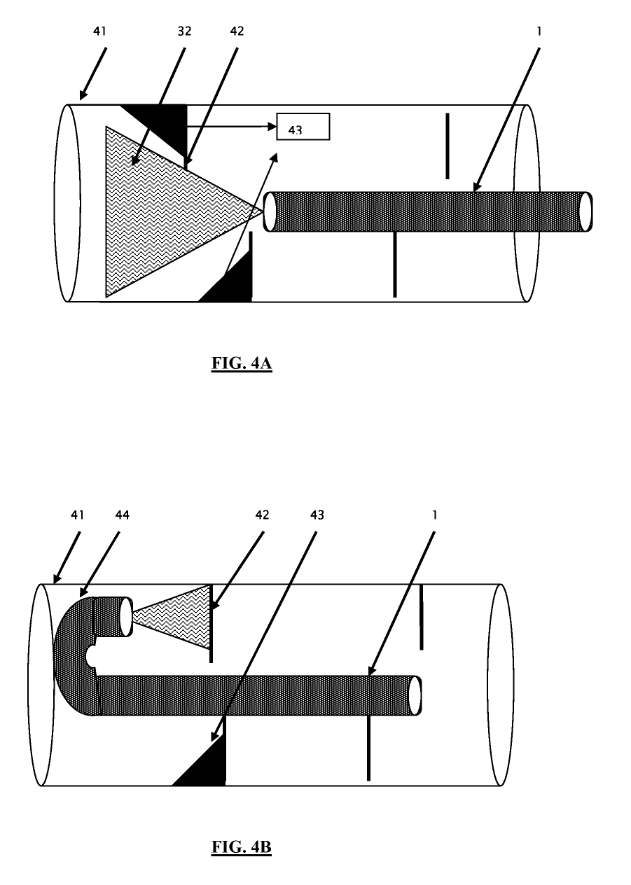

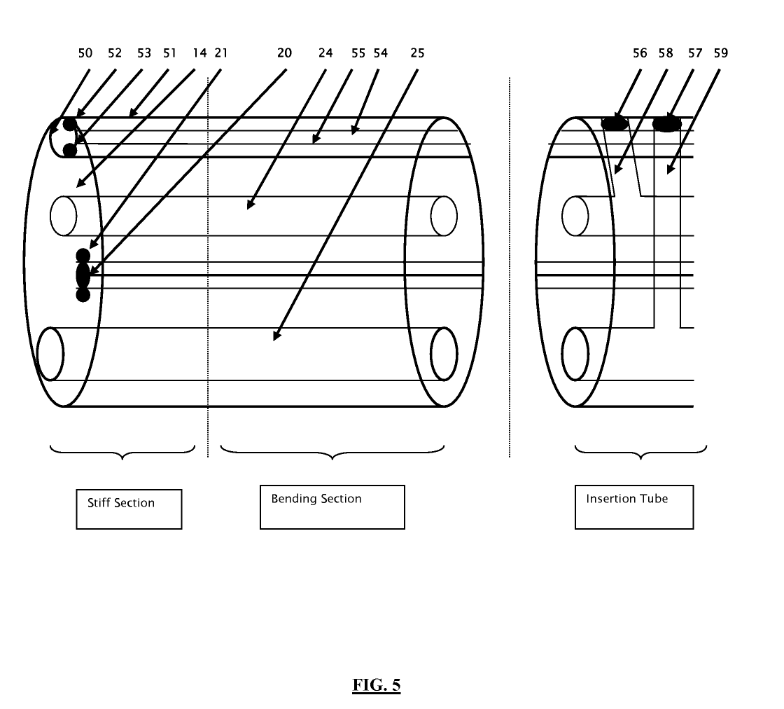

FIG. 4A shows side view of an endoscope (1) inside colon (41). The colon has mucosal folds (42). The front side of a mucosal fold blocks the view of the areas behind it during a typical endoscopic examination. These areas form the ‘blind spots’ (43) of a conventional endoscope that lie outside of the forward field of vision (32).

FIG. 4B shows side view of the rear flexion maneuver (44) of a conventional endoscope (1) inside colon (41). During this maneuver, the endoscope is advanced beyond the mucosal fold (42) to be examined. The bending portion of the endoscope is then bent to 180 degrees to visualize the rear side of a mucosal fold (43) during forward examination, the view of which is obstructed by its front side during a forward examination.

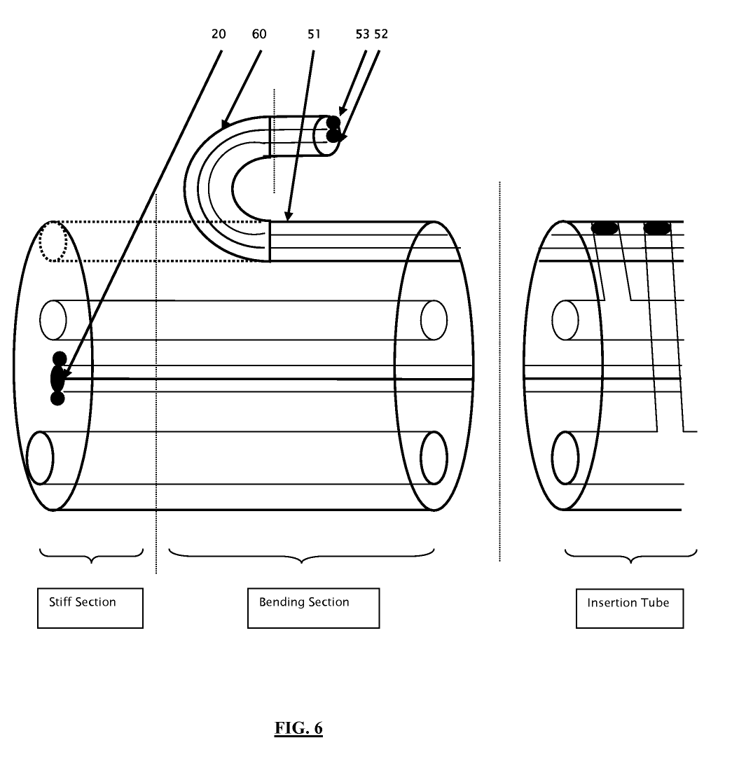

FIG. 5 shows side view of a first preferred embodiment of the present invention. The rear view module (51) is a long thin tubular structure encased in a sheath. It is placed along the periphery of a conventional endoscope. In the preferred embodiment, the rear view module (51) extends through the entire length of the endoscope but it may be shorter. The rear view module (51) has a distal end (50), stiff section, bending section and proximal section similar to an endoscope. In the preferred embodiment, the distal end (50), stiff section, bending section and proximal section of the rear view module (51) is in sync with the distal end (14), stiff section, bending section and proximal section of a conventional endoscope. The distal end (50) of the rear view module has a rear image lens (52) and a rear illumination bulb (53). The rear image lens (52) is connected to an image processor (not shown) and the rear illumination bulb (53) is connected to a power source (not shown) by electrical cables (54, 55) that run within the rear view module (51). Two pairs of cables within the rear view module attach the bending section of the rear view module to a rear view module actuator. Tension on these cables moves the bending section of the rear view module in vertical and horizontal planes. In the preferred embodiment, there is a rear air/water channel (58) with a rear air/water nozzle (56) and rear instrument channel (59) with a rear instrument channel outlet (57) located proximal to the bending section of the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (59) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. FIG. 6 shows the preferred embodiment in FIG. 5 where the rear view module (51) is rear flexed (60) using the rear view module actuator. With this maneuver, the rear image lens (52) faces backward and provides a rear view. The rear illumination bulb (53) illuminates the area under view of the rear image lens (52). The main image lens of the endoscope (20) provides a front view at the same time when the rear image lens (52) is providing a rear view. However, the operator may choose to have only one view at a given time. Because the rear view module is thin, rear flexion can be achieved with a small radius of curvature and thus can be performed even inside narrow hollow organs.

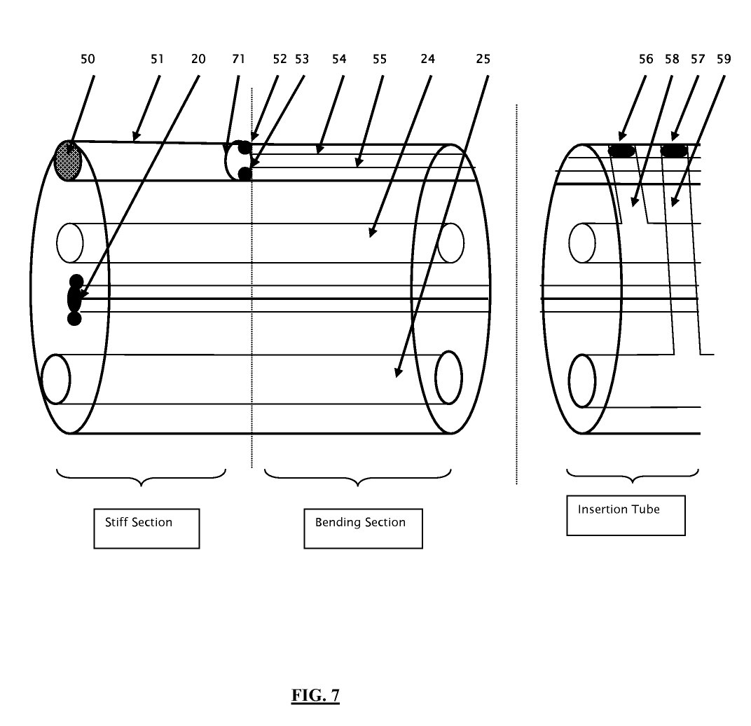

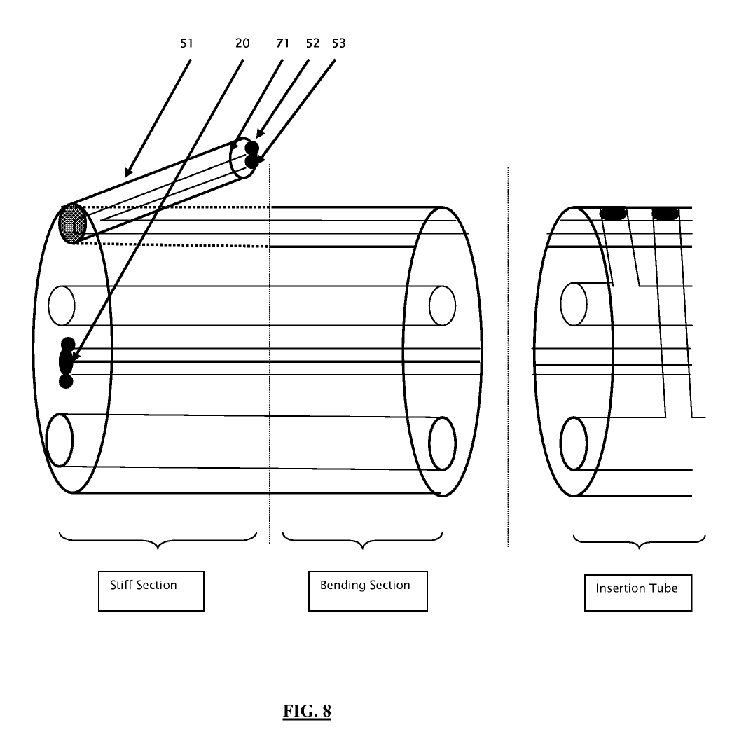

FIG. 7 shows side view of a second preferred embodiment of the present invention. The rear view module (51) is a solid rectangular block with a proximal end (71) and a distal end (50). It is located within the stiff section of the endoscope. The rear image lens (52) and the rear illumination bulb (53) are located on the proximal end (71) of the rear view module. The rear image lens (52) is connected to an image processor and the rear illumination bulb (53) is connected to a power source by electric cables (54, 55). The distal end (50) of the rear view module is attached to the distal end (14) of the endoscope by a hinge joint or any other suitable mechanical articulation. The distal end (50) of the rear view module is connected to a rear view module actuator by a pair of cables (not shown). Tension on these cables moves the rear view module away from and towards the shaft of the endoscope as shown in FIG. 8. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. FIG. 8 is a side view of the endoscope in FIG. 7 where the rear view module (51) has been deployed by lifting its proximal end (71) away from the shaft using the rear view module actuator. When fully deployed, the rear image lens (52) and the rear illumination bulb (53) face backward. The image captured by the rear image lens (52) is transmitted to an image processor. The rear illumination bulb (53) illuminates the area under view of the rear image lens (52). The main image lens (20) is able to give a forward view at the same time as the rear image lens is giving a rear view. Forward and rear view can thus be obtained simultaneously if so desired by the operator. A major advantage of this embodiment is that it makes rear view possible requiring only minimal additional space. This is of particular advantage when examining narrow body cavities.

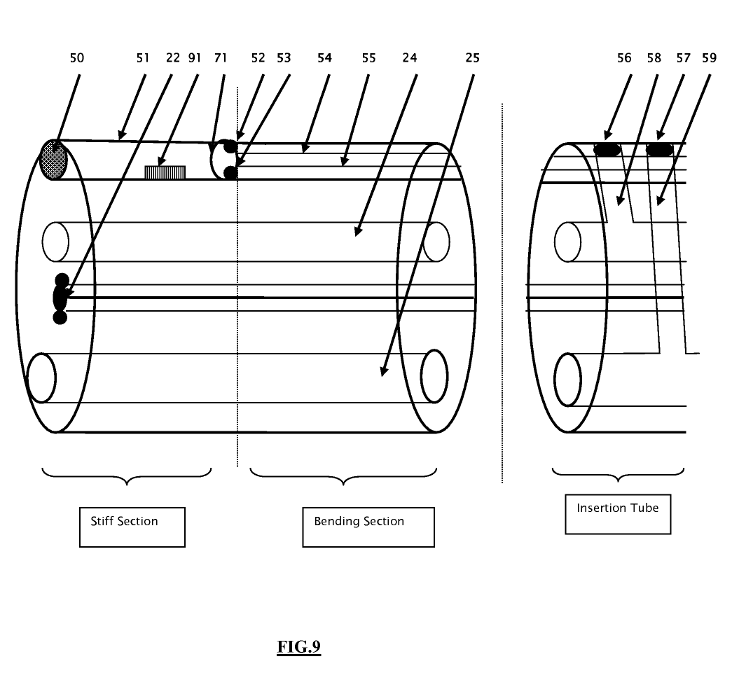

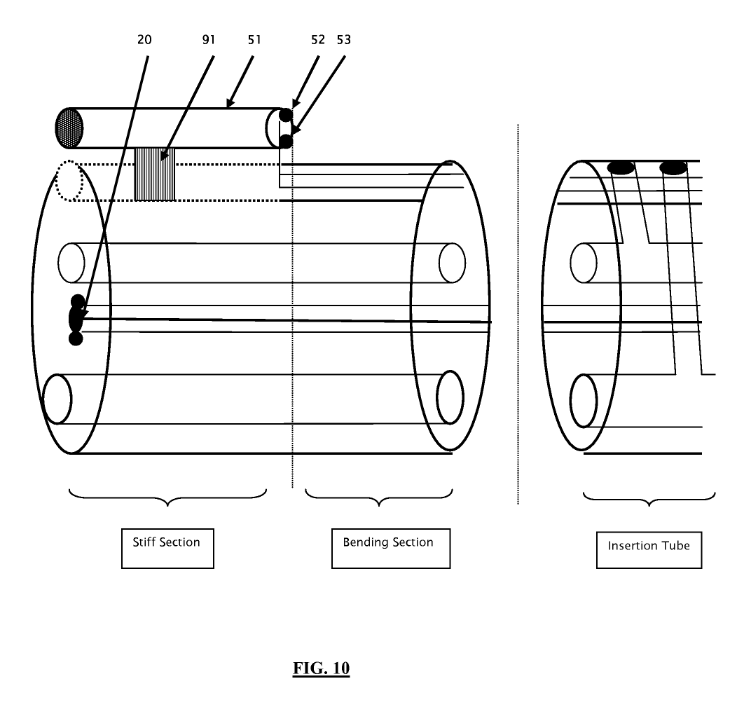

FIG. 9 shows side view of a third preferred embodiment of the present invention. The rear view module (51) is a solid rectangular block with a proximal (71) and distal (50) end. It is located within the stiff section of the endoscope. The rear image lens (52) and the rear illumination bulb (53) are placed on the proximal end (71) of the rear view module. The rear image lens (52) is connected to an image processor and the rear illumination bulb (53) in connected to a power source by electric cables (54, 55). The rear view module rests on a support pillar/spring (91). The support pillar/spring can be extended and retracted perpendicular to the shaft of the endoscope. It is attached to a rear view module actuator by cables. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. FIG. 10 shows the endoscope in FIG. 9 where it has been deployed by moving the support pillar/spring (91) vertically from the shaft using the rear view module actuator. In this position, the rear image lens (52) and the rear illumination bulb (53) face backward. The rear image lens (52) provides a rear view and the rear illumination bulb (53) illuminates the area under the view of the rear image lens (52). The main image lens (20) is able to provide a forward view at the same time when the rear image lens (52) is providing a rear view. This enables simultaneous forward and rear view if so chosen by the operator. A major advantage of this embodiment is that it provides a straight rear view that is desirable for certain surgical procedures.

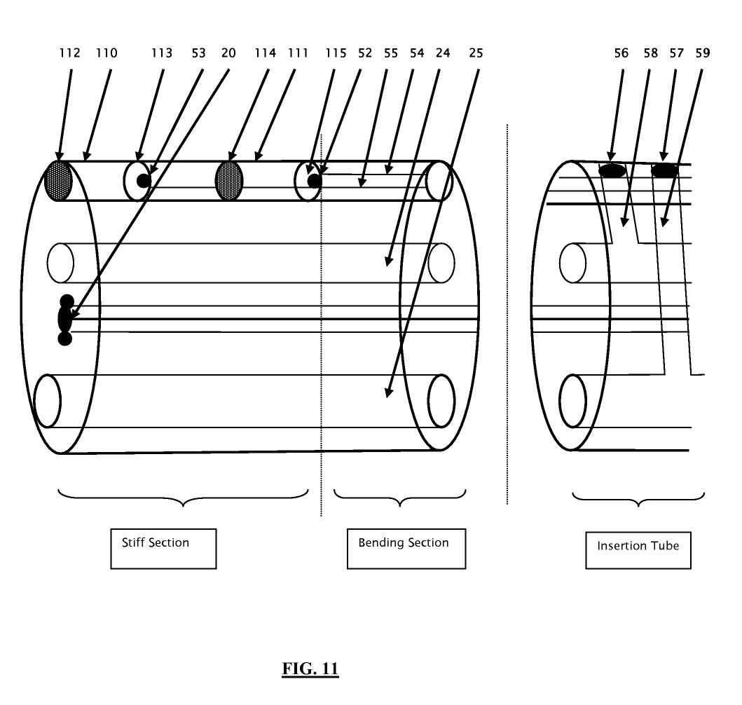

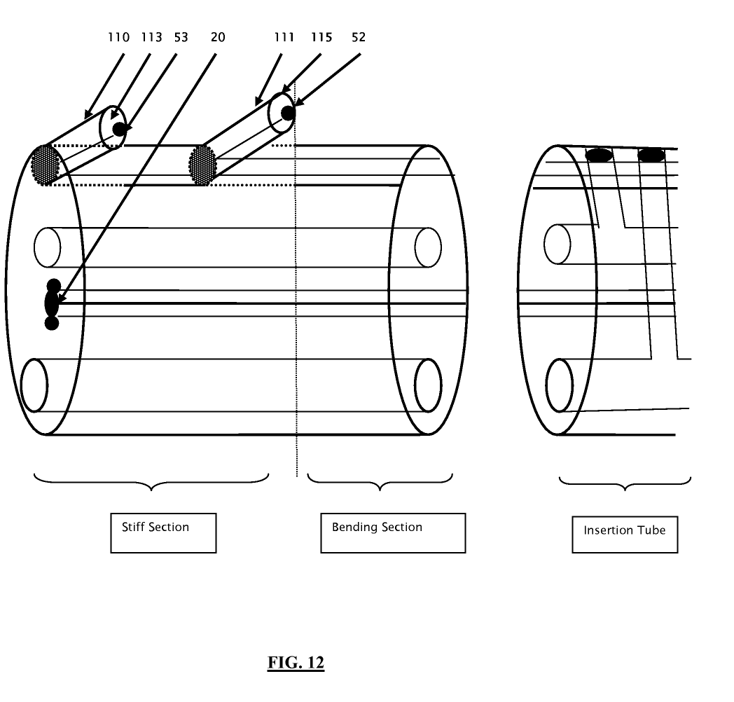

FIG. 11 shows a side view of a fourth preferred embodiment of the present invention. The rear view module (51) is made of two sub modules, the rear image module (111) and the rear illumination module (110). The sub modules are small rectangular solid structures. They are placed within the stiff section of the endoscope. The rear image module contains the rear image lens (52) and the rear illumination module contains the rear illumination bulb (53). The rear image lens (52) is placed on the proximal end (115) of the rear image module (110) and the rear illumination bulb (53) is placed on the proximal end (113) of the rear illumination module (111). The rear image lens (52) is connected to an image processor by an electric cable (54) and the rear illumination bulb (53) is connected to a power source by an electric cable (55). In the preferred embodiment, the rear image module (111) and the rear illumination module (110) are embedded within the stiff section of the endoscope. The distal end (114) of the rear image module and the distal end (112) of the rear illumination module are attached to the shaft of the endoscope by a hinge joint or any other suitable mechanical articulation. The distal ends of the rear image module and of the rear illumination module (112, 114) are also connected to a pair of rear view module actuators by cables. Tension on these cables moves the rear image module (111) and rear illumination module (110) away from and towards the shaft as shown in FIG. 12. The rear image module (111) and the rear illumination module (110) are placed at a suitable distance from each other. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. According to another aspect of the preferred embodiment the relative positions of the rear illumination module and the rear image module can be interchanged. According to another aspect of the preferred embodiment more than one rear illumination module and/or rear image module can be present. FIG. 12 is a side view of the endoscope in FIG. 11 where the rear image module (111) and the rear illumination module (110) have been deployed by moving their proximal ends (113,115) away from the shaft using the rear view module actuators. In this position the rear image lens (52) and the rear illumination bulb (53) face backward and provide a rear view. The main image lens (20) is able to provide a front view at the same time when the rear image lens is providing a rear view thus enabling a simultaneous front and rear view. A major advantage of the preferred embodiment is that the rear illumination module (110) can be controlled independent of the rear image module (111). This may be desirable in certain situations.

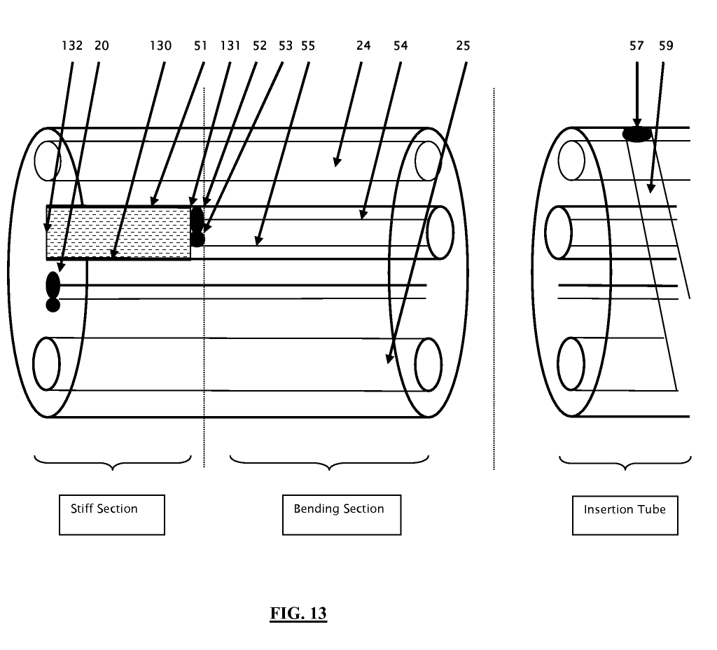

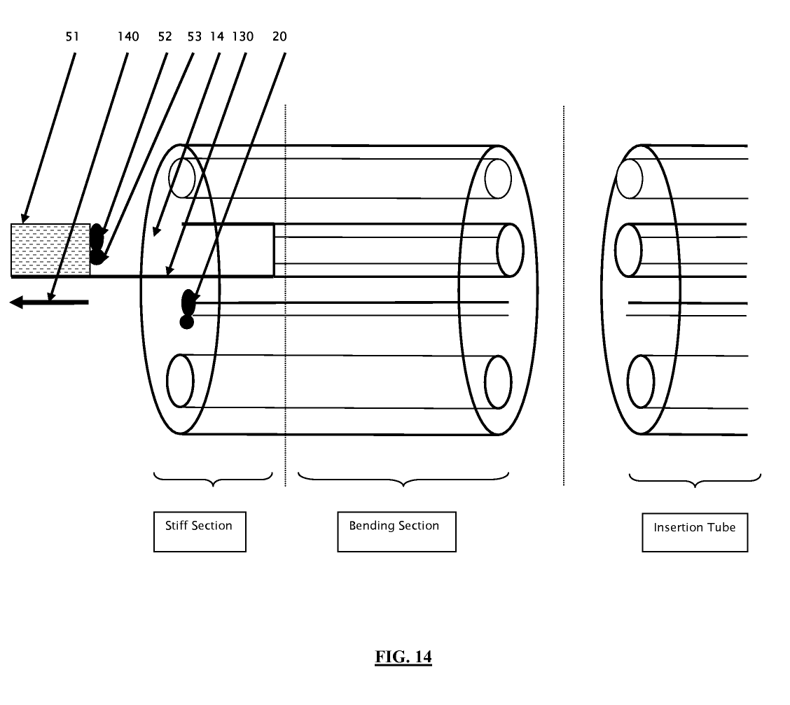

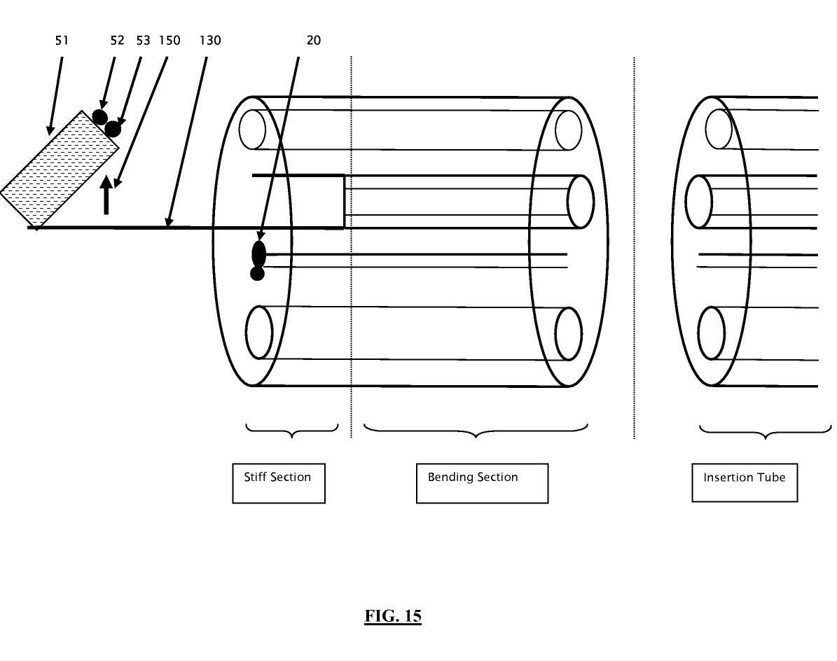

FIG. 13 shows a side view of a fifth preferred embodiment of the present invention. The rear view module (51) is a solid rectangular block with a proximal (131) and distal ends (132). It is located within the stiff section of the endoscope. It contains a rear image lens (52) and a rear illumination bulb (53) placed on the proximal end (131) of the rear view module. The rear image lens (52) is connected to an image processor by an electric cable (54). The rear illumination bulb (53) is connected to the power source by an electric cable (55). The rear view module (51) rests on a support arm (130) within the stiff section of the endoscope. The support arm (130) also serves as an extension arm that can be extended, retracted and rotated. The distal end (132) of the rear view module is attached to the support arm (130) by a hinge joint or any other suitable mechanical articulation. It is also connected to a rear view module actuator by cables. Tension on these cables moves the rear view module (51) away from and towards the support arm (130). In the preferred embodiment, there is a rear instrument channel (59) with a rear instrument channel outlet (57) located proximal to the rear view module (51). It is used to pass surgical instruments to do various surgical procedures in areas under view of the rear image lens (52). It is also used to direct suction to the area under the view of the rear image lens (52). The main air/water channel (24) is used to clean the rear image lens (52) and the rear illumination bulb (53). In the preferred embodiment, the rear instrument channel (59) is connected to the main instrument channel (25). However, it may exist independently. Passage to the rear instrument channel (59) from the main instrument channel (25) is controlled by a valve or any other suitable mechanical device. Typically, deployment of the rear view module (51) will automatically open the passage to the rear instrument channel (59). Alternatively, the passageway can be controlled independently. In the preferred embodiment, the rear view module is served by the main air/water channel (24). However a rear air/water channel may be provided. According to another aspect of the preferred embodiment, an additional forward image lens and an additional forward illumination bulb can be present at the distal end (132) of the rear view module. This will widen the forward field of vision. According to another aspect of the present invention, more than one rear view module can be present. FIGS. 14-16 shows side view of the endoscope in FIG. 13 where the rear view module (51) has been deployed for rear view. The support arm (130) is extended forward (140) to an appropriate distance from the distal end (14) of the endoscope as shown in FIG. 14. The rear image lens (52) faces backward in this position and gives a rear view. The rear illumination bulb (53) faces backward and illuminates the area under view of the rear image lens (52). The main image lens (20) is able to give a front view at the same time as the rear image lens (52) is giving a rear view, thus enabling simultaneous front and rear views. The rear view module (51) can be lifted from (150) and retracted towards the support arm (130) using the rear view module actuator as shown in FIG. 15. In addition, the support arm can be rotated (160) as shown in FIG. 16. This increases the rear field of vision.

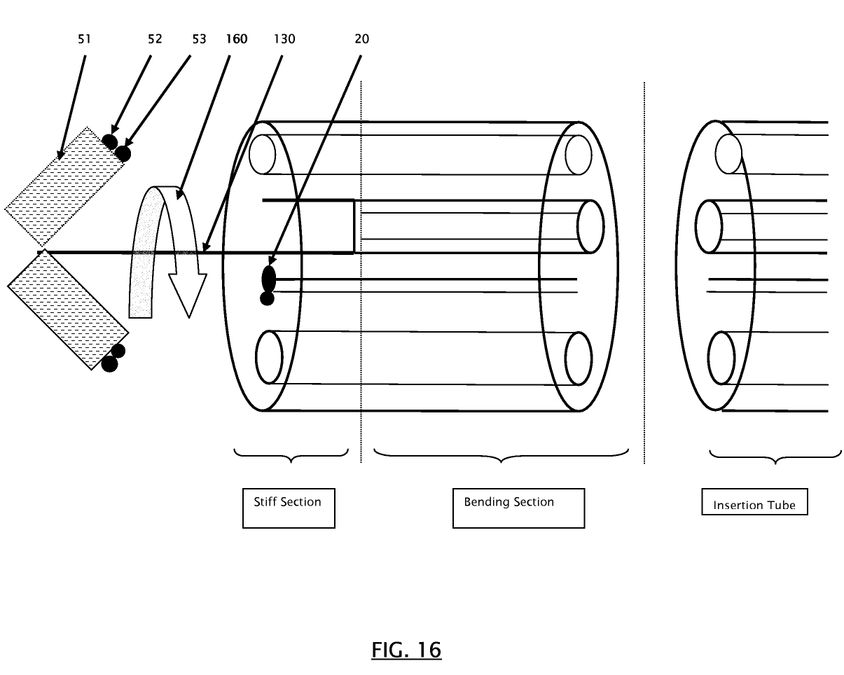

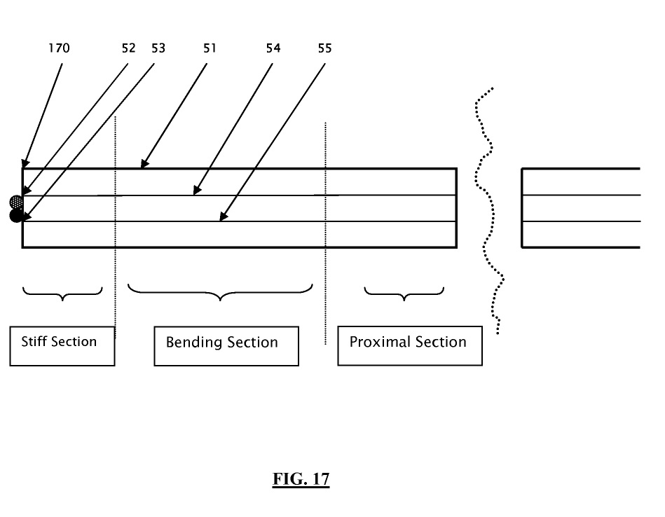

FIG. 17 shows side view of a sixth preferred embodiment of the present invention. The rear view module (51) is a long and thin tubular structure encased in a sheath. It has a shaft that comprises of a distal end (170), stiff section, bending section and proximal section. The shaft is attached proximally to a handle (not shown). The handle has an extension that connects the rear view module (51) to an image processor and a power source. Rear image lens (52) and rear illumination bulb (53) are placed on the distal end (170) of the rear view module (51). The rear image lens (52) and the rear illumination bulb (53) are connected to an image processor and a power source respectively by electrical cables (54, 55). The bending section of the rear view module is connected to a rear view module actuator by cables. Tension on these cables moves the bending section in vertical and horizontal planes. This entire assembly is thin enough to pass through the main instrument channel (25) of the endoscope. The rear view module (51) is passed through the instrument channel (25) beyond the distal end (14) of the endoscope as shown in FIG. 18. Backward view is obtained by rear flexing (190) the bending portion of the rear view module (51) as shown in FIG. 19. According to another method, bending section of rear view module comprises of shape memory material which urges the distal tip of the rear view module in rear viewing position upon exit from instrument channel of endoscope. In this position, the rear image lens (52) and the rear illumination bulb (53) face backward. The rear image lens (52) gives a rear view and the rear illumination bulb (53) illuminates the area under the view of the rear image lens (52). The main image lens (20) is able to give a forward view at the same time as the rear image lens (52) is giving a rear view. Simultaneous forward and rear view can thus be obtained if desired by the operator. The rear image lens (52) and the rear illumination bulb (53) are serviced by the main air/water channel (24). In a variation of the preferred embodiment, it can have a rear air/water channel and/or a rear instrument channel. In another variation to the preferred embodiment, the rear view module (51) can be passed through the rear instrument channel if one is present. In another variation of the preferred embodiment, the rear view module is embedded within the shaft of the endoscope. It is extended beyond the distal end of the endoscope and then rear flexed / bent to give a rear view.

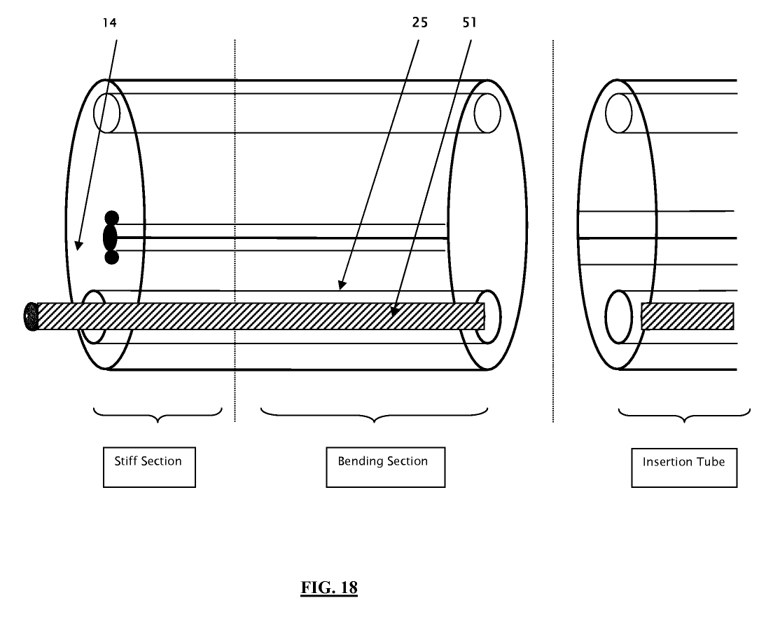

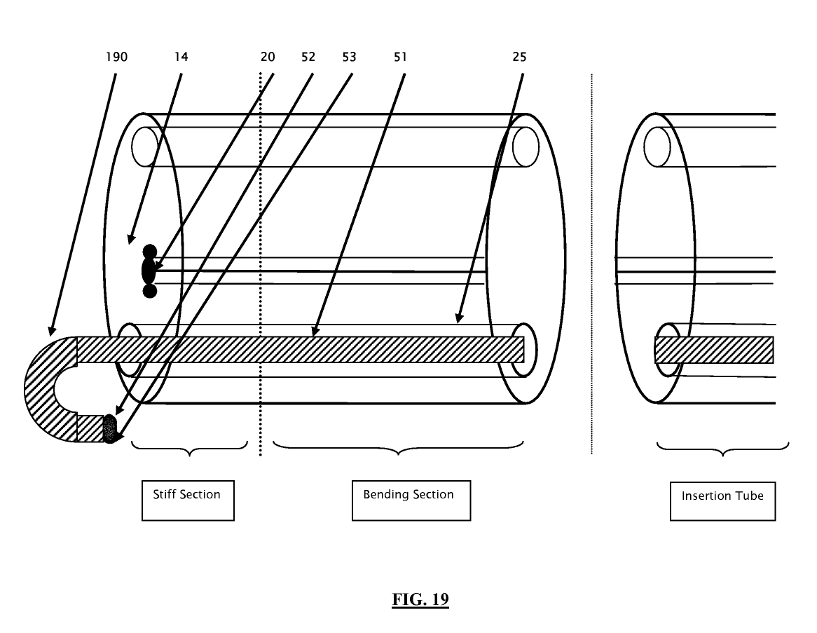

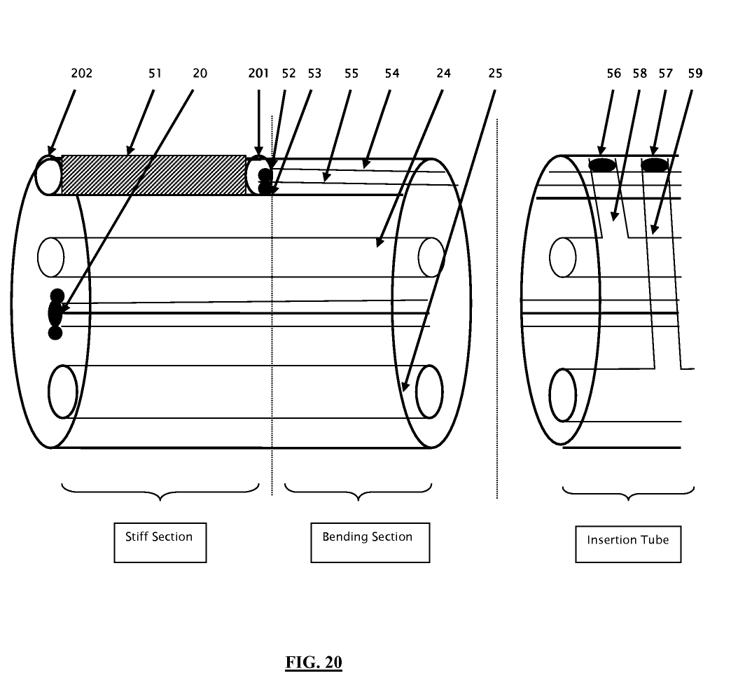

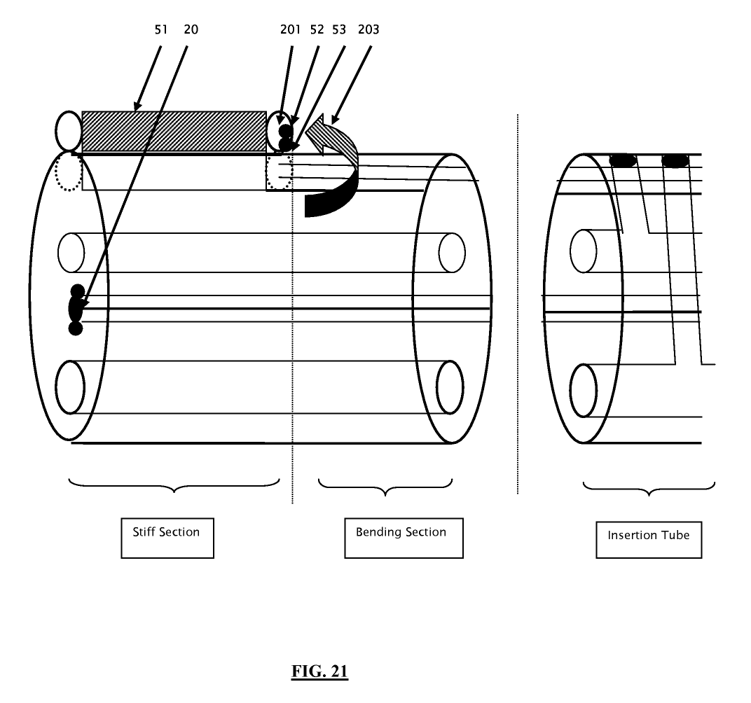

FIG. 20 shows side view of a seventh preferred embodiment of the present invention. The rear view module (51) is a hollow tubular structure with a proximal end (201) and a distal end (202). It is placed within the peripheral part of the stiff section of the endoscope, parallel to its long axis. The rear view module (51) is connected along its length to the stiff section of the endoscope by a hinge joint or any other suitable mechanical articulation. The rear image lens (52) and the rear illumination bulb (53) are placed on the proximal end (201) of the rear view module. The rear image lens (52) is connected to an image processor and the rear illumination bulb (53) is connected to a power source by electric cables (54, 55). Two pairs of cables one on the outside and the other on the inside, connect the rear view module to an actuator along its length. Tension on these cables opens and closes the module like the lid of a box (203) as shown in FIG. 21. When opened, the rear image lens (52) and the rear illumination bulb (53) face backward. The rear image lens (52) gives a rear view and the rear illumination bulb (53) illuminates the area under view of the rear image lens (52). The main image lens (20) of the endoscope is able to give a forward view at the same time as the rear image lens (52) is giving a rear view. Hence, simultaneous forward and rear view is possible if the operator so desires. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently.

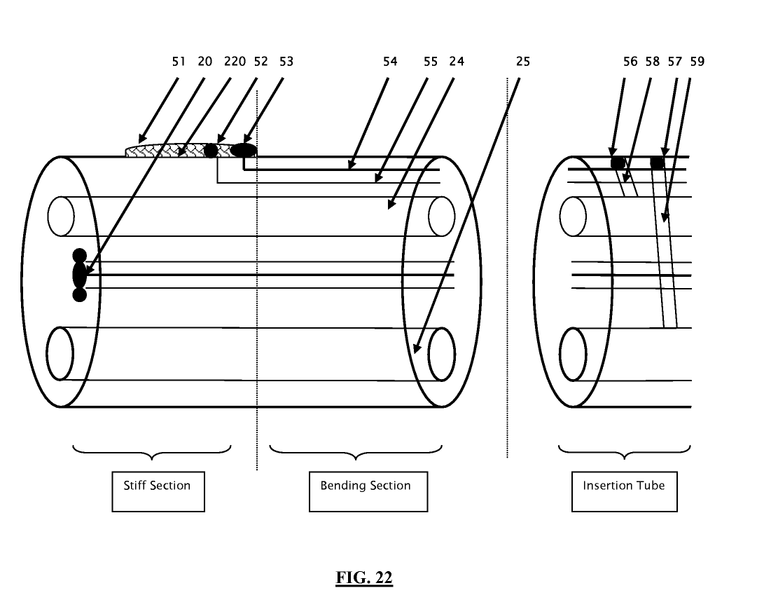

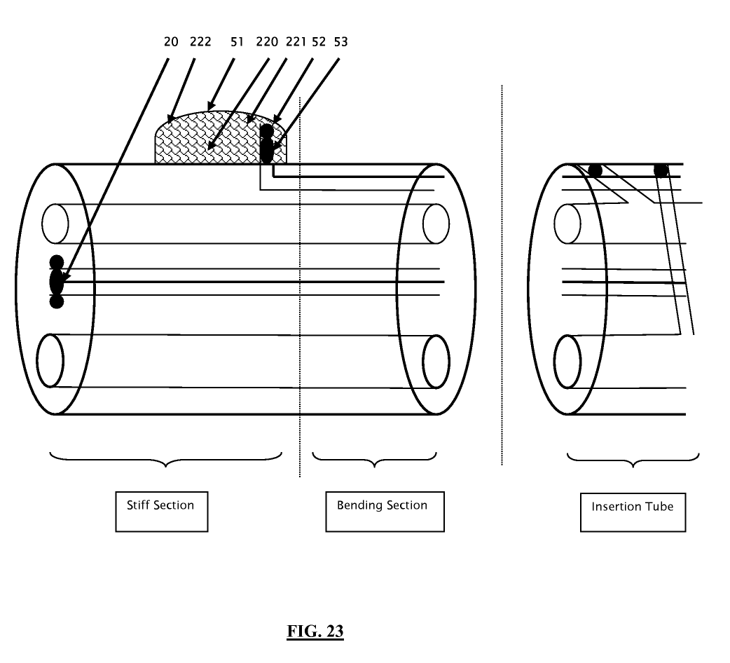

FIG. 22 shows side view of an eighth preferred embodiment of the present invention. The rear view module (51) consists of an inflatable balloon (220) or any other inflatable device that is attached to the stiff section of the endoscope. The balloon is connected to an air pump by a thin tube placed within the shaft of the endoscope (not shown). When inflated, the balloon (220) has a proximal face (221) and a distal face (222) as shown in FIG. 23. The proximal face (221) of the balloon contains the rear image lens (52) and the rear illumination bulb (53). Electric cables (54, 55) connect the rear image lens (52) to an image processor and the rear illumination bulb (53) to a power source. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument (59). Alternatively, the passageways can be controlled independently. Inflating the balloon (220) deploys the rear view module as shown in FIG. 23. When the balloon is fully inflated, the rear image lens (52) and the rear illumination bulb (53) face backwards. The rear image lens (52) gives a rear view and the rear illumination bulb (53) illuminates the area under view of the rear image lens (52). The main image lens (20) of the endoscope is able to give a forward view at the same time as the rear image lens (52) is giving a rear view. Hence, simultaneous forward and rear view is possible if the operator desires so. In a variation to the present embodiment, there can be an additional forward image lens and an additional forward illumination bulb placed on the distal face (222) of the balloon. This will widen the forward field of vision when the balloon (220) is inflated.

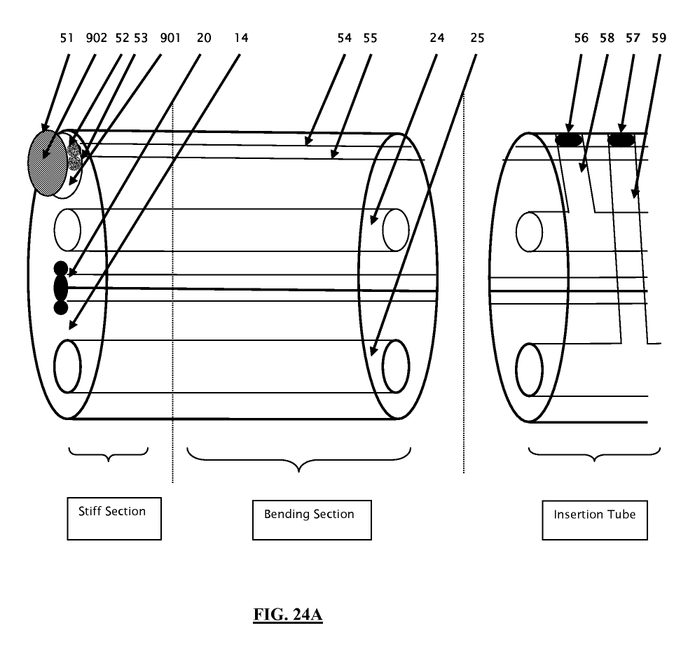

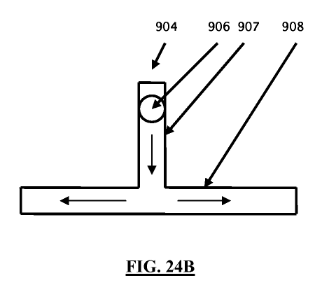

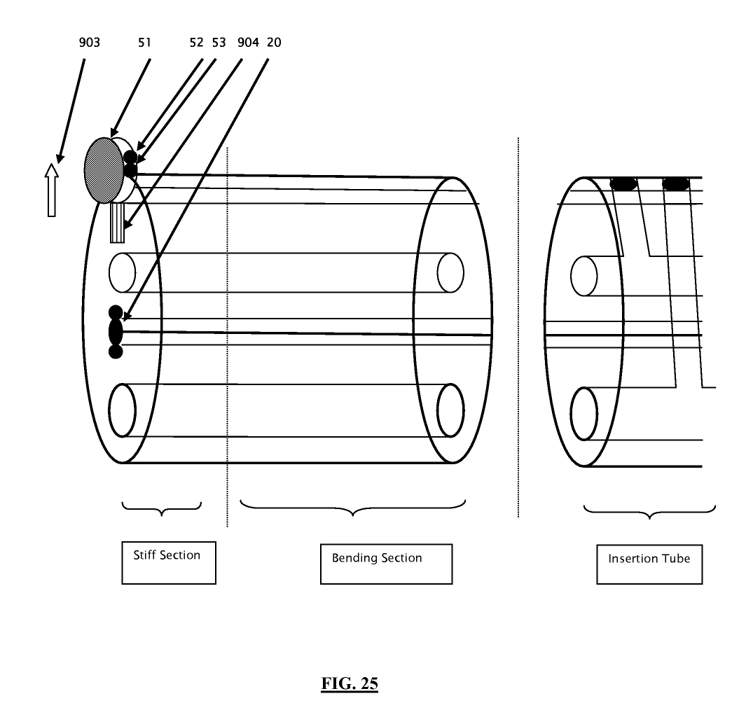

FIG. 24A shows side view of a ninth preferred embodiment of the present invention. The rear view module (51) is a solid disc shaped structure that has a proximal face (901) and distal face (902). It is mounted on the distal end (14) of the endoscope. It comprises of a rear image lens (52) and a rear illumination bulb (53) that is placed on the proximal face (901). The rear image lens (52) is connected to an image processor and the rear illumination bulb (53) is connected to a power source by electrical cables (54, 55). In the preferred embodiment, the rear view module (51) is placed towards the periphery of the distal end (14) of the endoscope but it may be placed at anywhere on the distal end (14). The proximal face (901) of the rear view module is attached to the distal end (14) of the endoscope by a bi planar rolling joint (904) as shown in FIG. 24B. It allows rolling motion of the rear view module in both vertical and horizontal planes from the distal end (14). Alternatively, the rear view module may be attached using any other suitable mechanical articulation. As shown in FIG. 24B, a bi planar rolling joint (904) consists of two grooves (907,908) placed orthogonally to each other. A small wheel (906) is placed within the groove. The outer part of this wheel is movable and the inner part is fixed. The rear view module (51) is attached to the fixed inner part. The rear view module is moved by rotating the wheel (906) along the grooves (907, 908). In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. The rear view module (51) is deployed by rolling it vertically (903) from the distal end of the endoscope (14), as shown in FIG. 25. Alternatively the rear view module can be deployed by rolling it horizontally from the distal end (14). The extent of the roll is enough to move the rear image lens (52) and the rear illumination bulb (53) away from the distal end of the endoscope (14). In this position, the rear image lens (52) gives a rear view and the rear illumination bulb (53) illuminates the area under view of the rear image lens (52). The rear view module (51) can also be rotated to widen the rear field of view. This may cause some distortion of the image which can be corrected by modifying the software of the image processor. The main image lens (20) of the endoscope is able to give a forward view at the same time as the rear image lens (52) is giving a rear view. Hence, simultaneous forward and rear view is possible if so desired by the operator. In a variation to the preferred embodiment the rear view module may contain an additional forward image lens and an additional forward illumination bulb on its distal face (902). This will widen the forward field of vision.

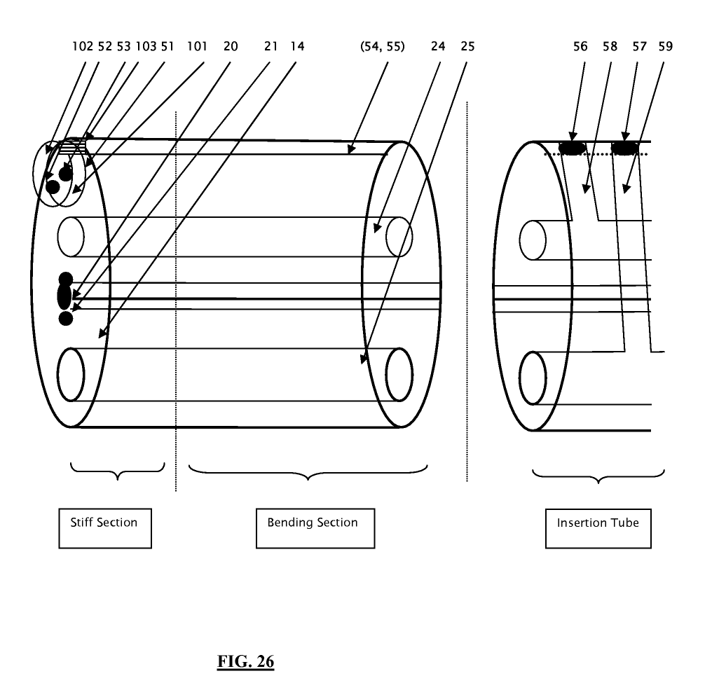

FIG. 26 shows side view of a tenth preferred embodiment of the present invention. The rear view module (51) is a solid discoid structure that is mounted on the distal end of the endoscope (14). It has a proximal face (101) and a distal face (102). The rear view module (51) is attached to the distal end of the endoscope (14) by a hinge joint (103) or any other suitable mechanical articulation. The rear view module (51) has a rear image lens (52) and a rear illumination bulb (53) that is mounted on its distal face (102) of the module. The rear image lens (52) is connected to an image processor and the rear illumination bulb (53) is connected to a power source by electrical cables (54, 55). In resting position, the rear image lens (52) and the rear illumination bulb face forward and augment the main image lens (20) and the main illumination bulb (21) to widen the forward field of view. In the preferred embodiment, the rear view module (51) is placed at the periphery of the distal end of the endoscope (14) but it can be placed anywhere. The rear view module (51) is connected to a rear view module actuator by cables. Tension on these cables flips the rear view module (51) clockwise and anticlockwise vertically from the distal end of the endoscope (14). Alternatively, the rear view module can be flipped in a horizontal plane. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. FIG. 27 shows the endoscope on FIG. 26 where the rear view module (51) has been deployed by flipping it vertically (104) from the distal end of the endoscope (14) to 180 degrees. In this position, the rear image lens (52) faces backward and gives a rear view. The rear illumination bulb (53) faces backward and illuminates the area under view of the rear image lens (52). The rear view module (51) can also be rotated in different directions to widen the rear field of vision. The main image lens (20) of the endoscope is able to give a forward view at the same time as the rear image lens (52) is giving a rear view. Hence, simultaneous forward and rear view is possible if the operator so desires. In a variation to the preferred embodiment, the rear view module (51) may also contain an additional forward image lens and an additional forward illumination bulb on its proximal face (101). This will increase the forward field of vision when the rear view module is deployed (104) with its proximal face (101) facing forward.

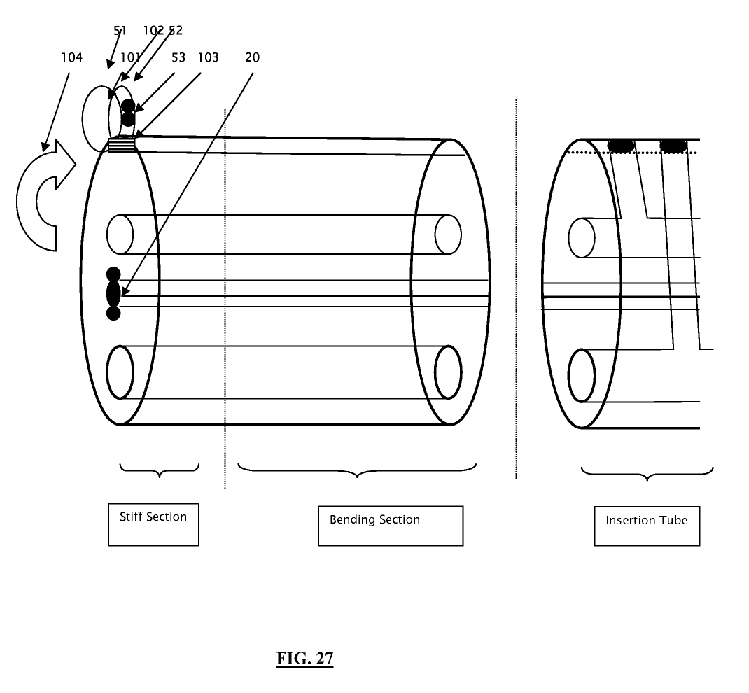

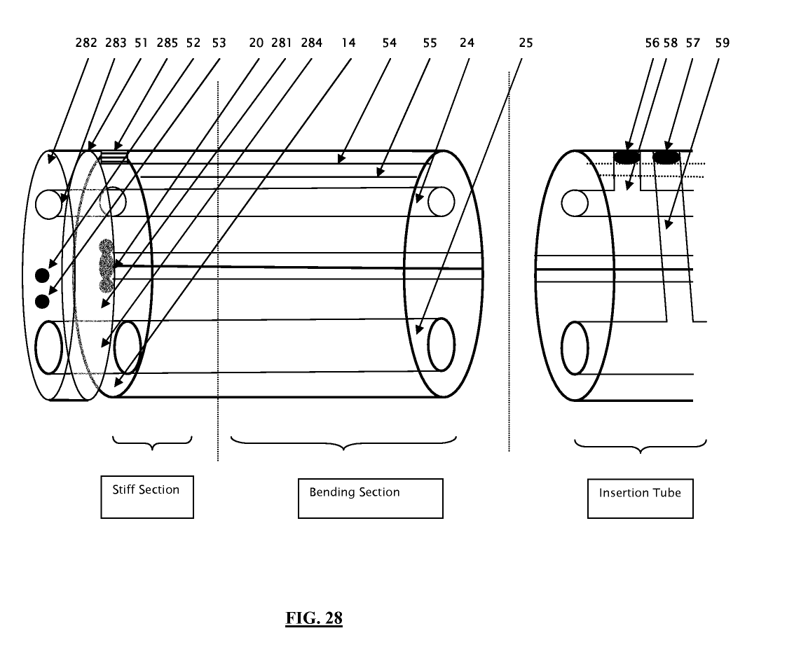

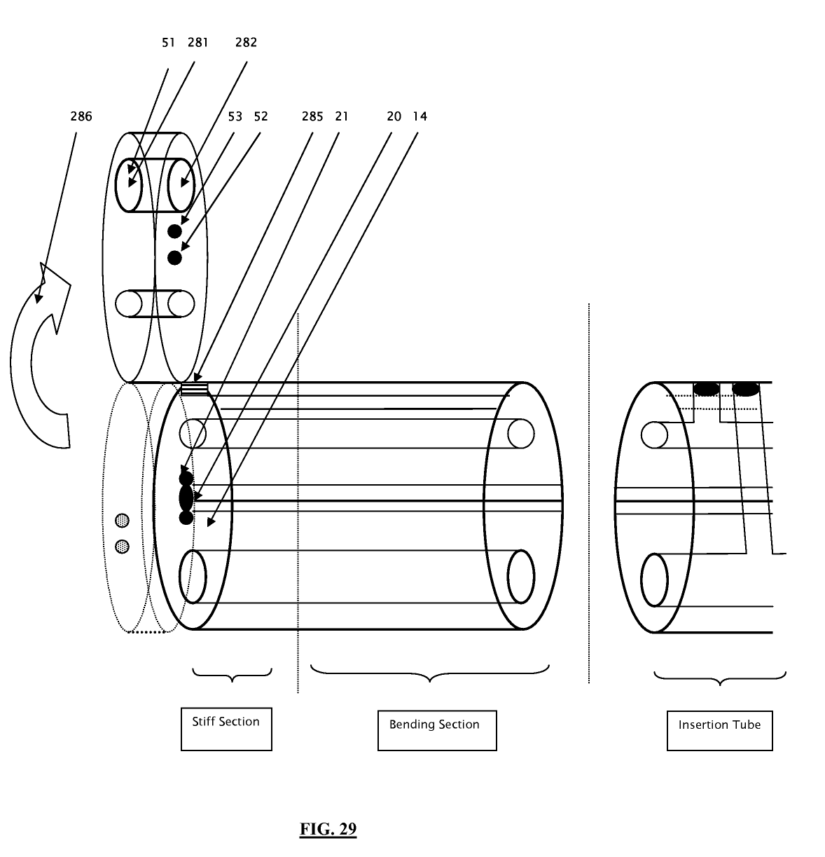

FIG. 28 shows side view of an eleventh preferred embodiment of the present invention. The rear view module (51) is a solid discoid structure that is placed in front of the distal end (14) of the endoscope. The periphery of the rear view module (51) is attached to the distal end (14) of the endoscope by a hinge joint (285) or any other suitable mechanical articulation. It has a proximal face (281) and a distal face (282). The rear image lens (52) and the rear illumination bulb (53) are placed on the distal face (282) of the rear view module. The rear image lens (52) is connected to an image processor and the rear illumination bulb (53) is connected to a power source by electric cables (54, 55). In resting position, the rear view module (51) covers the distal end of the endoscope (14) and faces forward. In this position, the rear image lens (52) gives a forward view and the rear illumination bulb (53) illuminates the area in front of the endoscope. In the preferred embodiment, the diameter of the rear view module (51) is the same as that of the distal end of the endoscope (14). The air/water channel (24) and the instrument channel (25) of the endoscope extend into the rear view module (283,284). The proximal and distal face of the rear view module (281, 282) is connected to a rear view module actuator by cables. Tension on these cables flips the rear view module (51) clockwise and anti clockwise vertically from the distal end of the endoscope (14) as shown in FIG. 29. Alternatively, the rear view module can be flipped horizontally. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. FIG. 29 shows the endoscope in FIG. 28 where the rear view module (51) has been deployed by flipping it vertically from the distal end of the endoscope to 180 degrees (286) using the rear view module actuator. In this position, the rear image lens (52) and the rear illumination bulb (53) face backward. The rear image lens (52) gives a rear view and the rear illumination bulb (53) illuminates the area under view of the rear image lens (52). Further, the rear view module (51) can be rotated in this position to increase the rear field of view. Upon deployment, the rear view module (51) moves away from the front of the distal end (14) of the endoscope. It enables the main image lens (20) to give a forward view and the main illumination bulb (21) to illuminate the area in front of the distal end of the endoscope. This makes it possible to have simultaneous forward and rear view if so desired by the operator. In a variation to the preferred embodiment, the rear view module contains an additional forward image lens and an additional forward illumination bulb on its proximal face (281). When the rear view module is deployed, the proximal face (281) with the additional forward image lens and additional illumination bulb faces forward and augments the main image lens (20) and the main illumination bulb (21. This widens the forward field of vision when the rear view module is deployed.

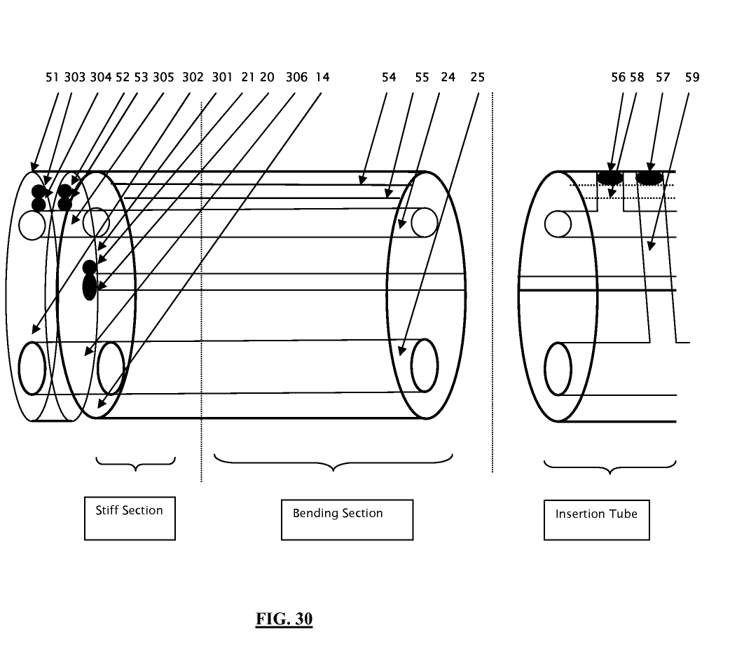

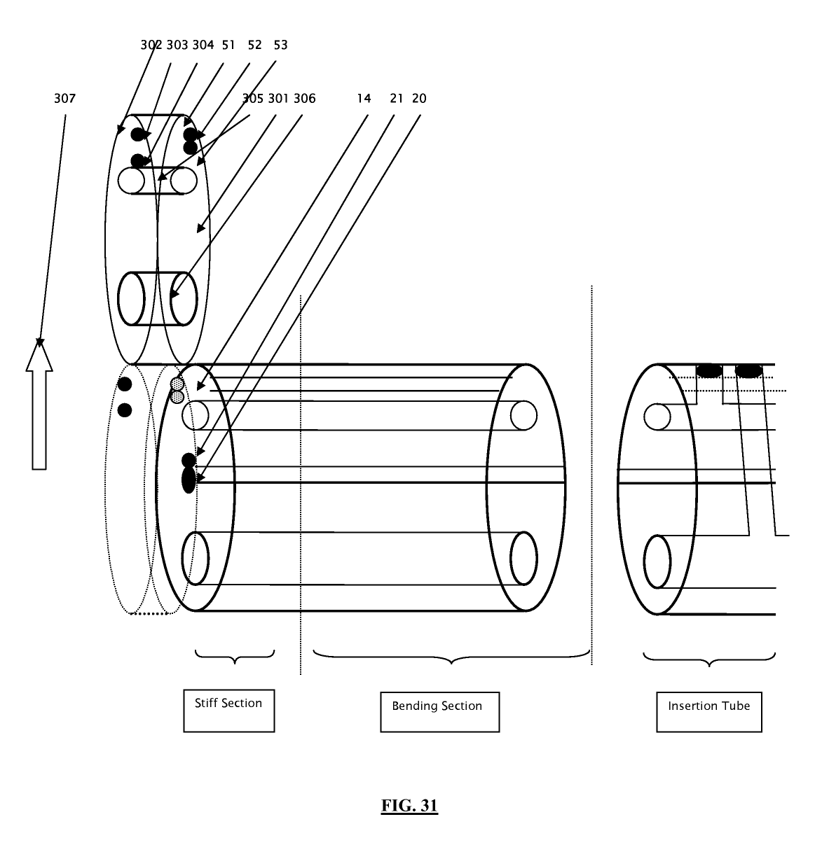

FIG. 30 shows side view of a twelfth preferred embodiment of the present invention. The rear view module (51) is a solid discoid structure that is placed in front of the distal end of the endoscope (14). It has a proximal face (301) and a distal face (302). The rear view module comprises of a rear image lens (52) connected to an image processor and a rear illumination bulb (53) connected to a power source by electric cables (54, 55). The rear image lens (52) and the rear illumination bulb (53) are placed on the proximal face (301) of the rear view module (51). In addition, the rear view module (51) has an additional image lens (303) and an additional illumination bulb (304) that is placed on its distal face (302). In the preferred embodiment, the diameter of the rear view module (51) is the same as that of the distal end of the endoscope (14). The main air/water channel (24) and the main instrument channel (25) of the endoscope extend into the rear view module (305, 306). The rear view module (51) is attached to the distal end of the endoscope (14) by a bi planar rolling joint as shown in FIG. 24B. This allows rolling motion of the rear view module (51) both vertically and horizontally to the distal end of the endoscope (14). It may also be attached by any other suitable mechanical articulation. In resting position, the rear view module (51) covers the main image lens (20) and the main illumination bulb (21) of the endoscope. In this position, the additional image lens (303) and the additional illumination bulb (304) faces forward and gives a forward view and illuminates the area in front of the endoscope. In the preferred embodiment, there is a rear air/water channel (58) with a rear air/water nozzle (56) and rear instrument channel (59) with a rear instrument channel outlet (57) located proximal to the rear view module (51). The rear air/water channel (58) provides a jet of water and a stream of air that is used to clean the rear image lens (52) and the rear illumination bulb (53). It is also used to insufflate air in the field of vision of the rear image lens (52) for better distension and visualization. Surgical instruments are passed through the rear instrument channel (25) to do various surgical procedures in the area under view of the rear image lens (52). It is also used to direct suction to the area under the view of the rear image lens (52). In the preferred embodiment, the rear air/water channel (58) and the rear instrument channel (59) is connected to the main air/water channel (24) and the main instrument channel (25) respectively. However, these may exist independently. Passage to the rear air/water channel (58) and rear instrument channel (59) from the main air/water channel (24) and main instrument channel (25) is controlled by a valve or any other suitable mechanical device. Typically, deployment of the rear view module (51) automatically opens the passage to the rear air/water channel (58) and the rear instrument channel (59). Alternatively, the passageways can be controlled independently. FIG. 31 shows the endoscope in the embodiment in FIG. 30 where the rear view module (51) has been deployed by sliding it vertically (307) from the distal end of the endoscope (14). Upon deployment, the rear image lens (52) and the rear illumination bulb (53) face backward. The rear image lens (52) gives a rear view and the rear illumination bulb (53) illuminates the area under view of the rear image lens (52). Further, the rear view module (51) can be rotated to increase the field of rear view. The rear view module (51) also moves away from front of the distal end of the endoscope (14) upon deployment. The main image lens (20) is then able to give a forward view and the main illumination bulb (21) is able to illuminate the area in front of the endoscope. Hence, the preferred embodiment provides simultaneous forward and rear view if so desired by the operator. The additional image lens (303) and the additional illumination bulb (304) augment the main image lens (20) and the main illumination bulb (21) and widen the forward field of vision when the rear view module (51) is deployed.

According to one aspect of the invention, means is provided to prevent interference between forward image lens and rear illumination bulb; and rear image lens and forward illumination bulb. This aspect is specifically relevant to embodiments of rear view module where the rear view module is positioned in front of the imaging assembly of the endoscope; such as the embodiments shown in FIGS. 13-16 & FIGS. 17-20. When the rear view module in this situation faces the distal tip of the endoscope, there is light interference between the forward illumination bulb and the rear image lens; and between the rear illumination bulb and the forward image lens. This impairs the clarity and quality of images obtained both from the forward and rear image lenses.

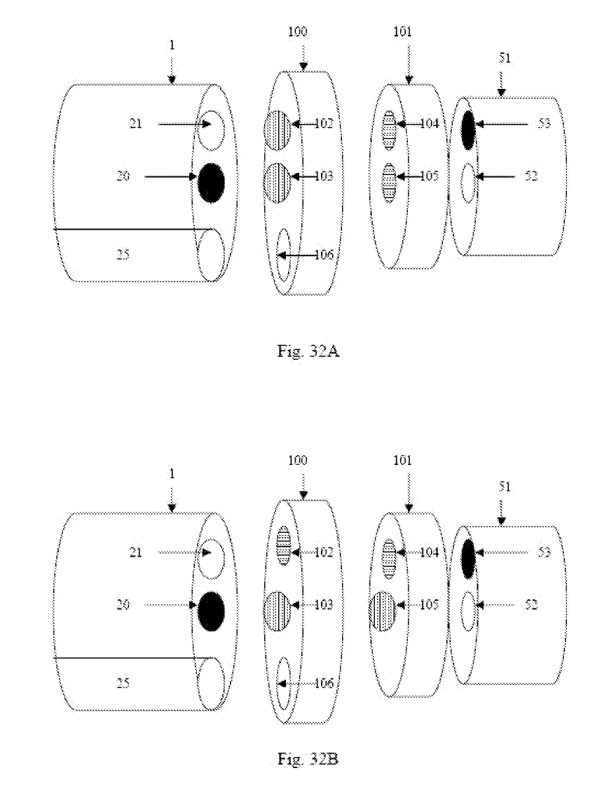

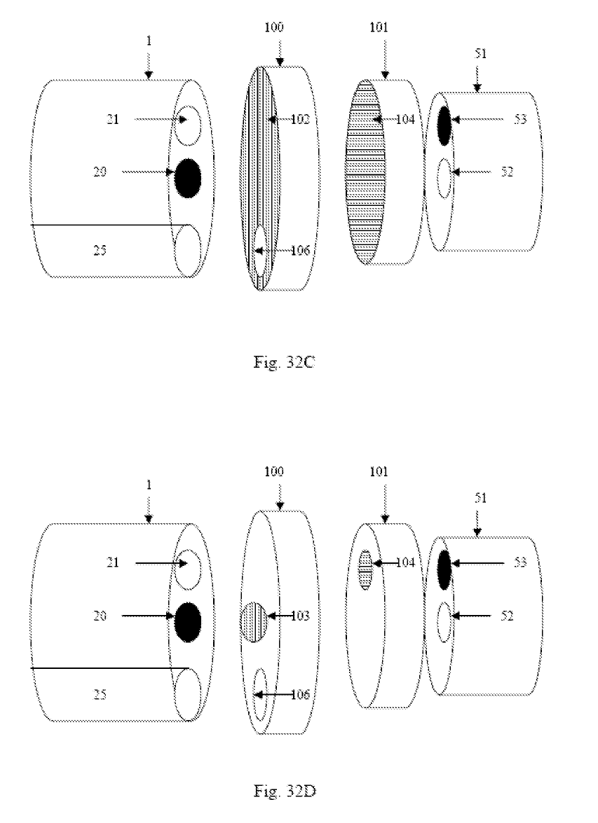

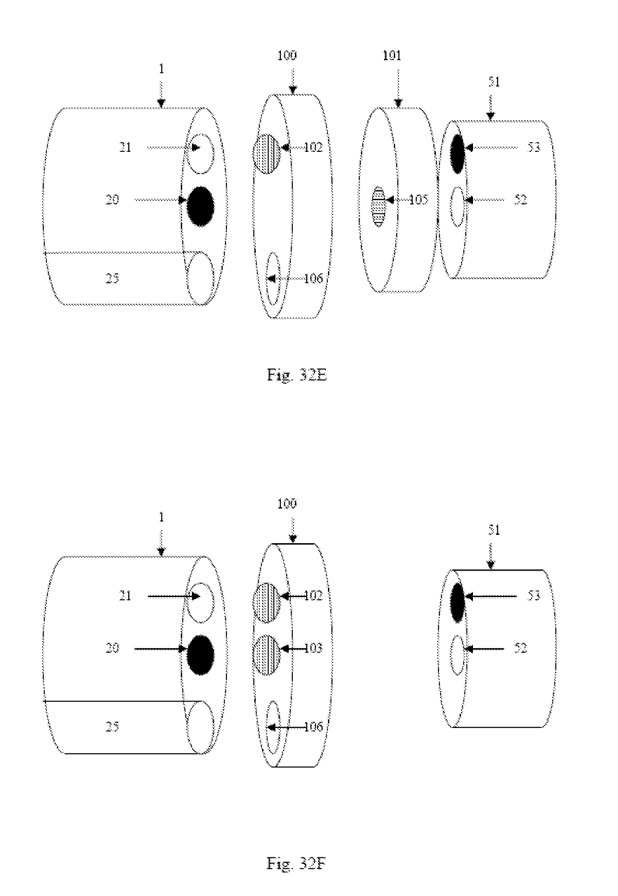

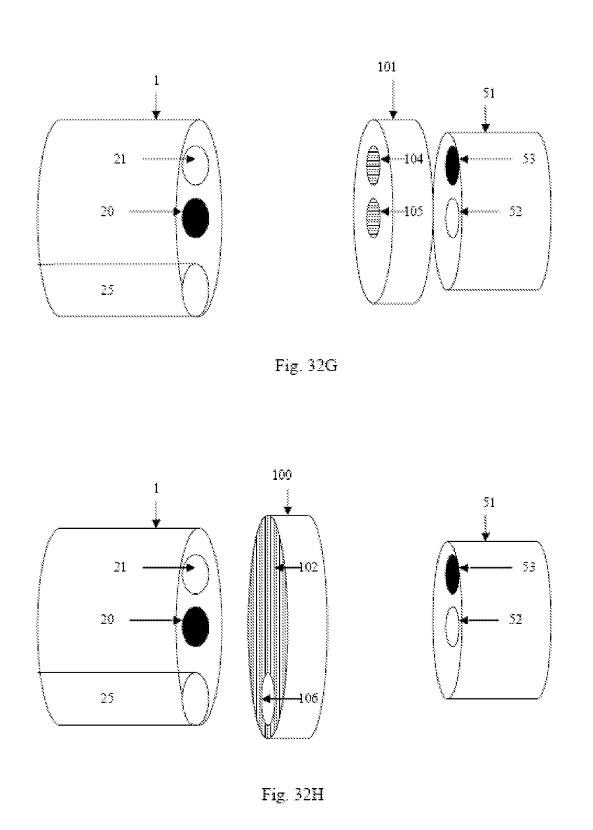

According to one method, linear or circular polarizing filters are used. Linear polarizing filter transmits light in one plane of polarization; and circular polarizing filter transmits light in only one particular orientation of polarization. Polarizing filter is preferably made from a material that enables light in one planar orientation to pass thorough while blocking light in other planes of orientation. Light blocking means in the polarizing filter may comprise one or more of reflection, scattering, absorption, birefringence, etc. When two polarizing filters are placed in front of each other the amount of light that passes through the two polarizing filters depend on the relative angle of orientation of the planes of the two filters. In case of arrangement of two linear polarizing filters facing each other, maximum passage of light happens when orientation is parallel; and minimum passage of light happens when the two filters are oriented at an angle of 90 degrees. Similarly, in case of two circular polarizing filters, minimum passage of light happens when it is a combination of right and left handed polarizing filters. In embodiments shown in FIGS. 32A-32I; linear polarizing filters are used. However, circular polarizing filter may as well be used in which case, preferably, a combination of right and left hand polarizing filters are used. In the discussion here after, linear polarizing filter is used as reference with an understanding that circular polarizing filters as disclosed above may as well achieve same end result. The polarizing filters are preferably attached using cap fitted onto the endoscope tip and rear view module. The polarizing filters are preferably placed in front of the image lens/illuminations source. Alternatively, the polarizing filter can be incorporated into the image lens/light source; can be placed directly onto the image sensor; can be placed anywhere in between the image lens and the image sensor; or can be placed anywhere between the light source and field of view. In the embodiments, light source is disposed at the light output port on the endoscope and rear view modules. However, it is to be understood light source can instead be disposed remote from the distal tip of the endoscope/rear view module; having light guide means between the light source and light output port at distal end of the endoscope/rear view module. Such an arrangement is known in prior art and does not merit further a detailed discussion.

According to first embodiment; as shown in FIG. 32A, a first polarizing filter pair (102, 103) with parallel orientation is placed in front of forward image lens (21) and forward illumination source (20). A second polarizing filter pair (104, 105) with parallel orientation is placed in front of rear image lens (52) and rear illumination source (53). The first polarizing filter pair (102, 103) is oriented as an angle of 90 degrees with respect to the second polarizing filter pair (104, 105). According to second embodiment, shown in FIG. 32B, using same principles of the first embodiment, a first polarizing filter pair (102, 103) with parallel orientation is placed in front of forward image lens (21) and rear illumination source (53). A second polarizing filter pair (104, 105) with parallel orientation is placed in front of rear image lens (52) and forward rear illumination source (53). The first polarizing filter pair (102, 103) oriented as an angle of 90 degrees with respect to the second polarizing filter pair (104, 105). According to third embodiment; shown in FIG. 32C, the forward image lens (21) and forward illumination source (20) share same first polarizing filter (102) mounted onto a cap (100) reciprocally fitted on to the endoscope tip. The rear image lens (52) and rear illumination source (53) share same second polarizing filter (104) is mounted onto a cap (101) reciprocally fitted on to the tip of the rear view module (51). This arrangement ensures that the polarizing planes are aligned for corresponding light source and image lens in the same plane; and enable optimal lighting conditions and image quality. The first polarizing filter (102) is oriented as an angle of 90 degrees with respect to the second polarizing filter (104). In embodiments shown in FIGS. 32A-32C, and as discussed above, opposing planes of orientation of polarizing light filters prevent light from forward and rear illumination source (20, 53) causing interference with respect to rear and forward image lens (52, 21). The arrangements of polarizing light filters described above enable forward image lens (21) to receive light from forward illumination source (20) while blocking interference from rear illumination source (53); thus improving forward image quality. Similarly, rear image lens (52) is able to receive light from rear illumination source (53) while blocking interference from forward illumination source (20) thus improving rear image quality.

According to fourth and fifth embodiments; shown in FIGS. 32D & 32E; polarizing filters are only used in front of image lens or only in front of illumination source. According to fourth embodiment shown in FIG. 32D, first polarizing filter (103) having first plane of orientation is placed in front of forward image lens (21); and second polarizing filter (104) having second plane of orientation is placed in front of rear image lens (52); with the first and second orientation planes at an angle of 90 degrees. According to fifth embodiment shown in FIG. 32E, first polarizing filter (104) having first plane of orientation is placed in front of forward illumination source (20); and second polarizing filter (105) having second plane of orientation is placed in front of rear illumination source (53); with the first and second orientation planes at an angle of 90 degrees. The perpendicular orientation of polarizing planes between forward and rear imaging lens in the embodiments shown in FIGS. 32D & 32E minimizes light entry into lens thereby minimizing light interference. The polarizing filters are fitted onto a cap (100, 101) reversibly attached to endoscope and rear view module.

According to sixth embodiment, shown in FIGS. 32F & 32G, only one pair of polarizing filter is used; as for example a pair of polarizing filters (103, 104) having parallel planes of orientation is placed in front of forward illumination source (20) and forward image lens (21). Using same principles, polarizing filters (104, 105) can alternatively be placed in front of rear image lens (52) and rear illumination source (53) as shown in the seventh embodiment in FIG. 32G. According to eight and ninth embodiments shown in FIGS. 32H & 321, one polarizing filter (102 &104) large enough to cover both the illumination source and image lens is provided in front of the forward and rear imaging assemblies respectively. In arrangement of polarizing filters as shown in FIGS. 32F-321; light entry into forward or rear image lens (21, 52) is minimized as a result of polarizing filters; and hence light interference is minimized.

According to second method of preventing interference; as shown in FIGS. 33A & 33B, means for automatic adjustment in light intensity of forward and rear illumination sources (20, 53) to achieve optimal light intensity in common view field is provided. Forward and/or rear illumination sources (20, 53) are connected to a light intensity control system (330); controlling quantity of illumination. Light intensity control system preferably comprises of light path diaphragm control system in conjunction with light intensity sensor chip. The forward and rear view modules (1, 51) are connected to video processor. Application software that runs on the video processor allows automatic synchronized adjustment of light intensity of the forward and rear illumination sources (20, 53) to achieve optimal light intensity in common field of view. In addition, optimum light density adjustment having means for automatic selection of the ideal sensitivity is provided. Further, automatic exposure control to capture photography having means to automatically set light source at the optimum exposure for photography, preferably using servo-diaphragm method, is provided. The forward and rear illumination sources (20, 53) preferably comprises of high-quality xenon lamp, which allows observation in deep sites or advanced techniques and can be used continuously for 500 hours. Additionally, image light exposure adjustment application (331) having means to adjust light intensity effect is provided in application software running on video processor. Alternatively, image quality and light exposure adjustment may be performed by hardware image chip sets or other electric circuitry. The image quality and light exposure adjustment techniques are well known in prior art and need not be described in great detail.

According to third method of preventing interference, means is provided for forward and rear illumination sources to provide light in different wavelengths. This light arrangement of forward and rear illumination sources minimizes interference. In one preferred method, forward illumination source provides white light and rear illumination source provides light in narrow wavelength band such as blue light (narrow band imaging).

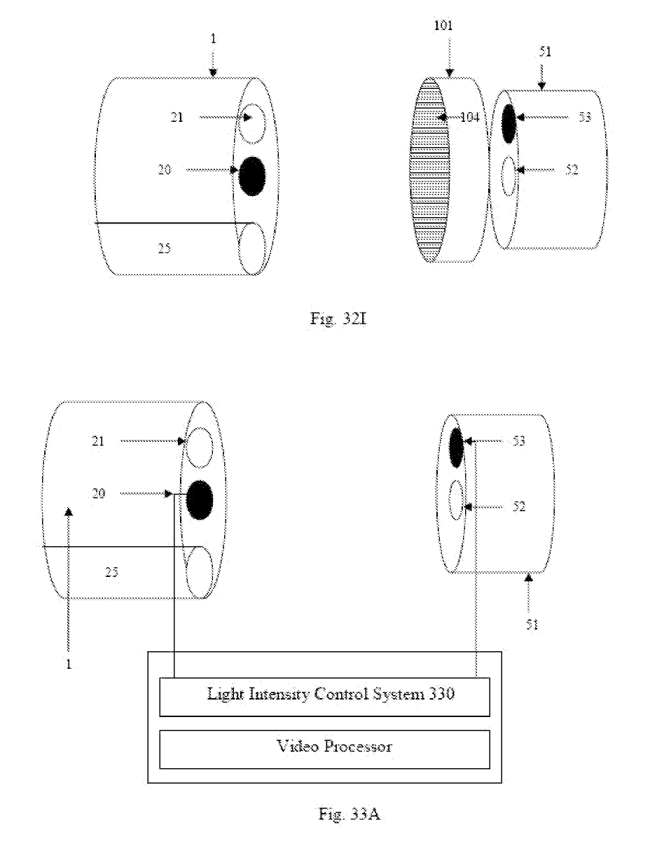

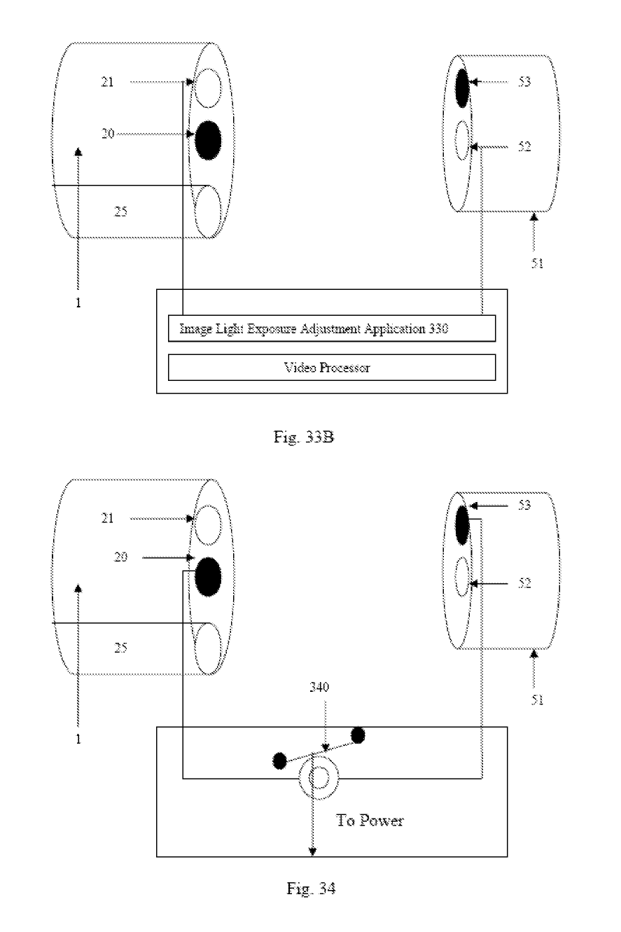

According to fourth method to prevent interference, as shown in FIG. 34, means is provided for alternate switching of forward and rear illumination sources. Forward and rear illumination sources (20, 53) are connected to an oscillator (340); which in turn is connected to power source. To complete electric circuit, forward and rear illumination sources (20, 53) have to be operatively connected live to the oscillator (340). Oscillator (340) is operatively connected to a motor (not shown) having means to move the oscillator (340) from first position to second position at high frequency, preferably greater than 60 hertz. In the first position, the oscillator (340) is connected to forward illumination source (20) and in the second position the oscillator (340) is connected to the rear illumination source (53). Preferably, the frequency of alternate switching of the forward and rear illumination sources (20, 53) is more than 60 hertz; at which frequency optical illusion is obtained and continuous simultaneous forward and rear view is determined by human eye. An advantage of this method is that there is no interference; as forward and rear illumination sources remain switched on alternatively and only one of the two sources remains switched on at a given time. It is to be understood instead of light sources disposed at the distal end of the endoscope and rear view module, light source can be provided remote from the distal tip of the endoscope/rear view module; and light guide means may be provided between the light source and light output port on distal end of the endoscope/rear view module.

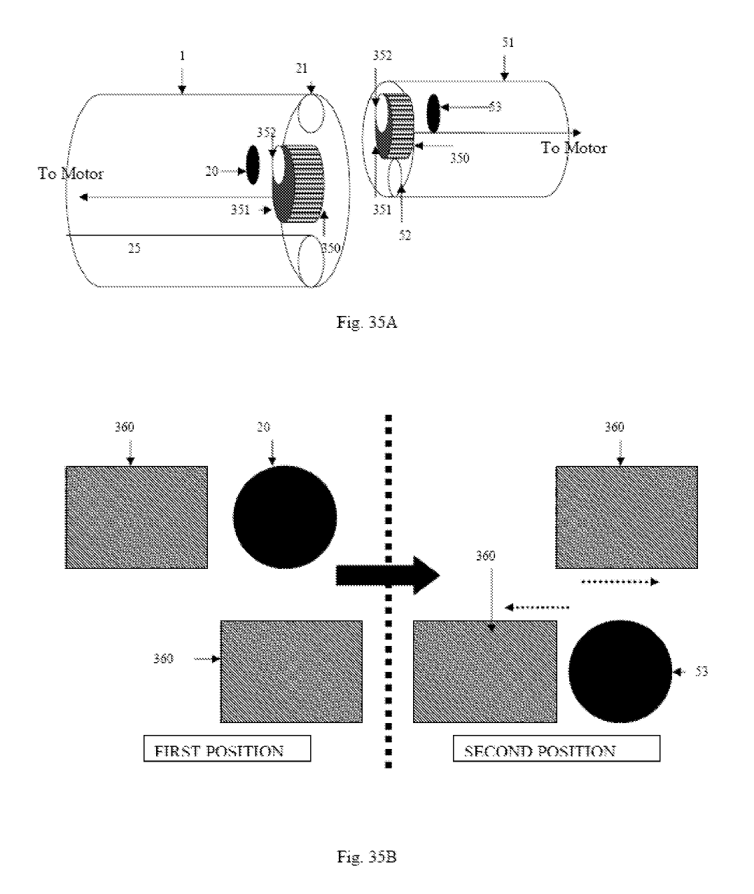

According to fifth method to prevent interference, shown in FIG. 35A, light screen means having means to block light alternatively from forward and rear illumination sources at high frequency; preferably greater than 60 hertz is provided. Preferably, rotating disc (350) is provided in front of forward and rear view sources (20, 53). One half of the rotating disc is transparent (352) and the other half is opaque (351). The rotating discs (350) are operatively connected to motor (not shown) having means to rotate the discs in front of forward and rear illumination sources (20,53), preferably at a frequency of greater than 60 hertz. The rotation of the forward and rear rotating discs (350) with respect to corresponding illumination sources (20,53) is coordinated such that while transparent half (352) of first rotating disc is in front of forward illumination source (20), opaque half (351) of the second rotating disc is in front of rear illumination source (53). This enables alternate passage of light between first illumination and second illumination sources (20, 53). When rotating at high frequency, preferably greater than 60 hertz, the rotating discs (350) prevent interference of light between forward and rear illumination sources (20, 53); while providing simultaneous and continuous forward and rear views as a result of optical illusion to human eye. An advantage of this method is that it does not require high frequency activation and deactivation of illumination sources; hence improving their life span and efficiency. While rotating disc is described in the preferred embodiment as light screen; shape of the light screen means should not be considered limiting as light screens of other shapes and configurations can be used to achieve similar outcomes. For example, as illustrated in FIG. 35B, a rectangular shutters (360) operatively connected to motor providing means for alternate left to right movement of shutter (360) is provided in front of illumination sources (20, 53). When shutter (360) is in front of the source light passage is blocked. Movement of the shutters (360) is coordinated such that when first shutter (360) is in front the first illumination source (20), second shutter (360) is positioned away from the second illumination source (53), as shown in FIG. 35B.

Although in the above discussion, light screen means is provided in front of illumination sources disposed at the distal tip of the endoscope and rear view catheter; illumination sources may be disposed remotely from the endoscope and rear view catheter. Turning our attention to FIG. 36, it is well known in prior art relating to endoscopes that light source (20) can be remote from the distal tip of the endoscope. When so, light from remote light source (20) is conveyed to light output port at distal tip of the endoscope (362) by means of a light guide (361). Continuing from the principles discussed in FIGS. 35A & 35B, when endoscope system is constructed with remote light source, light screen means is provided in front of the remote light source. According to one preferred embodiment shown in FIG. 36, remote forward and rear light source (20, 53) is provided for the endoscope (1) and the rear view catheter (51) respectively. Light screen means comprising of rotating disc (350) is provided in front of remote forward and rear light sources (20, 53). One half of the rotating disc is transparent (352) and the other half is opaque (351). The rotating discs (350) are operatively connected to motor (not shown) having means to rotate the discs in front of remote forward and rear light sources (20,53), preferably at a frequency of greater than 60 hertz. The rotation of the forward and rear rotating discs (350) with respect to corresponding remote light source (20,53) is coordinated such that while transparent half (352) of first rotating disc is in front of forward light source (20), opaque half (351) of the second rotating disc is in front of rear light source (53). This enables alternate passage of light from forward and rear light sources (20, 53). Light emitted from forward and rear light source (20, 53) is carried to the distal tip of the endoscope (362) and the distal tip of the rear view catheter (364) respectively by means of their respective light guides (363, 365). When rotating at high frequency, preferably greater than 60 hertz, the rotating discs (350) prevent interference of light when distal tip of the rear view catheter (364) is facing the distal tip of the endoscope (362); while providing simultaneous and continuous forward and rear views; as a result of optical illusion to the human eye. An advantage of this method is that it does not require high frequency activation and deactivation of illumination sources; hence improving their life span and efficiency. Furthermore, construction and maintenance of this preferred embodiment is simpler on account of remote light source. It is to be understood that while rotating disc is described in the preferred embodiment as light screen; shape of the light screen means should not be considered limiting as light screens of other shapes and configurations can be used to achieve similar outcomes. For example, rectangular shutter can be used when endoscope and rear view catheter is constructed having remote light source, using the principles shown and discussed in FIG. 35B. First and second rectangular shutters operatively connected to motor providing means for alternate left to right movement of shutter is provided in front of forward and rear light source respectively. When shutter is in front of light source light passage is blocked. Movement of the shutters is coordinated such that when first shutter is in front the forward light source, second shutter is positioned away from the rear light source. The shutters are preferably moved at a frequency of grater than 60 hertz to obtain optical illusion to the human eye.

It is to be noted that all embodiments of the invention should be considered including the construction of the endoscope and rear view modules; wherein light source is remote from the distal tip of the endoscope/rear view module; and light guide means is provided between the light source and distal end of the endoscope/rear view module.

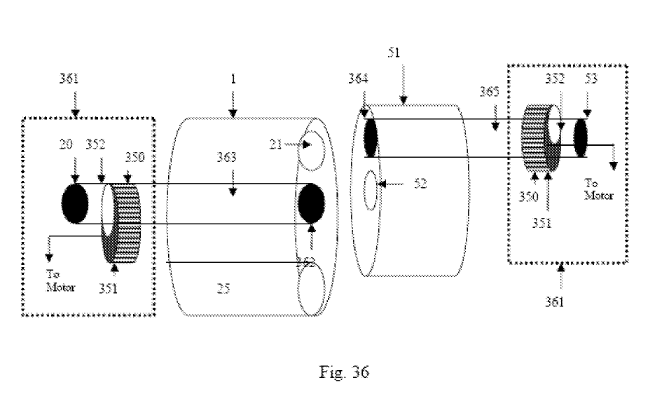

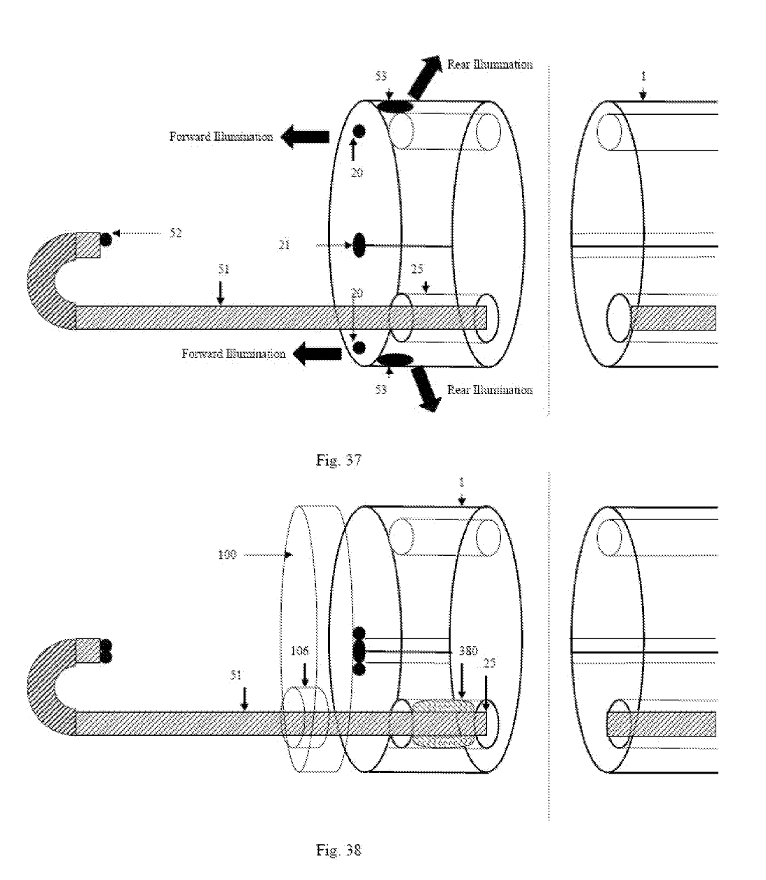

FIG. 37 shows a sixth method to prevent interference; especially as it relates to the embodiments of rear view modules as shown in FIGS. 13-16 & FIGS. 17-20; rear illumination source (53) is placed along the periphery of the distal end of the endoscope; proximal to the forward illumination source. Preferably, a receptacle is provide in the endoscope shaft to accommodate the rear illumination source (53); and the rear illumination source is focused away from the forward illumination source by an angle of greater than 120 degrees, as shown in FIG. 37. Rear view module (51) has image lens (52) disposed at its tip. This arrangement of forward and rear illumination sources (20, 53) prevent interference by virtue of their geographic location; whereby forward illumination source is placed in front of the rear illumination source and the rear illumination source faces backward with respect to the forward illumination source by an angle of more than 120 degrees. Preferably multiple rear illumination sources (53) are disposed along the periphery of the distal end of the endoscope. The rear illumination source (53) is connected to power source by electric cable running through the endoscope. In this arrangement; there is none or only minimal interference between the forward and rear illumination sources. According to an alternate arrangement; the rear facing rear illumination source is placed on a cap mounted on the tip of the endoscope; and positioned behind the forward illumination source. It is to be understood instead of light source disposed at the distal end of the endoscope and rear view module, light source can be provided remote from the distal tip of the endoscope/rear view module; and light guide means may be provided between the light source and distal end of the endoscope/rear view module.

According to another aspect of the invention, most relevant to the embodiment shown in FIGS. 17-20; means is provided to stabilize the rear view catheter in relation to the shaft of the endoscope. During our development we found that the rear view catheter when extended beyond the distal tip of the endoscope became unstable; and encountered uncontrollable and unwanted rotational movement with respect to the shaft of the endoscope. This impaired the image quality, especially around sharp turns of the colon. This was especially problematic when polarizing filters are applied to the rear view catheter and/or endoscope according to embodiments shown in FIGS. 32-35; as uncontrolled movement of the rear view catheter impaired the relative positions and workings of the polarizing filter arrangement.

According to first embodiment, as shown in FIG. 38, inflatable bladder (380) is attached to the outer periphery of the distal end of the rear view catheter (51). The inflatable bladder (380) is operatively connected to air or water pump by means of a thin hollow tube (not shown). Activation of the air/water pump inflates the bladder (380) to a desired volume. Preferably, the inflatable bladder (380) is made of pliable material with somewhat rough consistency such as unfinished rubber. During operation, the surgeon advances the rear view catheter (51) beyond the tip of the endoscope till retro flexed position of the tip of the rear view catheter housing image lens and illumination bulb is obtained. This is done by operation of the rear view catheter actuator. Alternatively, when bending portion of the rear view module is made of shape memory material, retro flexion of the rear view catheter tip is automatically obtained upon exit of the bending portion of the rear view catheter beyond the distal end of the endoscope. At this port, the inflatable bladder (380) is positioned inside the distal end of the hollow channel of the endoscope (25) or inside the hollow channel of the endoscope polarizing cap (106, 100). The bladder (380) is inflated using air/water till bladder till desired resistance is achieved; and there after the bladder is securely in opposition to the walls of the hollow channel of the endoscope (25)/endoscope polarizing cap (100). In this position, the bladder (380) securely anchors the rear view catheter inside the hollow channel of the endoscope (25)/endoscope polarizing cap (106) and effectively prevents rotational movement of the rear view catheter (51) with respect to the endoscope (1). Although in the above embodiment, only one inflatable bladder is disclosed, more than one inflatable bladder may be used. According to another variation of the first embodiment; the inflatable bladder (380) is placed inside the hollow channel of the endoscope (25) or inside the hollow channel (106) of the endoscope polarizing cap (100), while achieving the same end result with respect to stabilizing the rear view catheter.

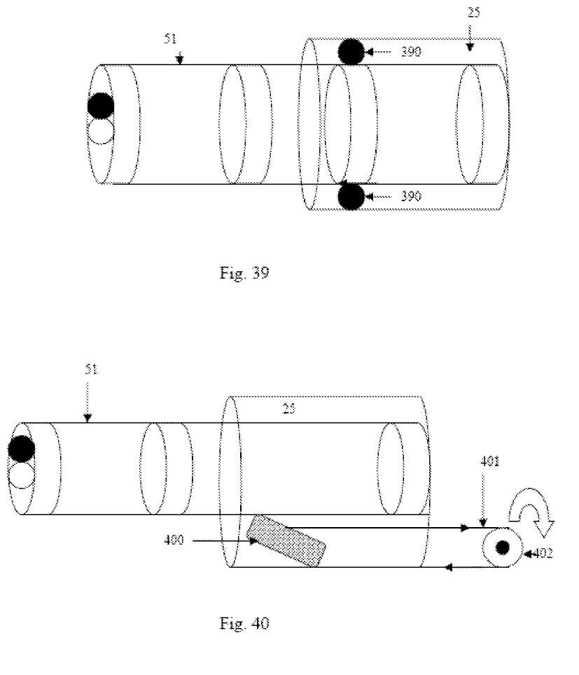

According to second embodiment, as shown in FIG. 39, resistance methods such as compressive beads (390) are provided inside the distal end of the hollow channel of the endoscope (25)/endoscope polarizing cap. The resistance beads (391) are preferably made of soft pliable material such as rubber, soft plastic etc. The resistance beads (390) are positioned circumferentially along the inner surface of distal end of the hollow channel of the endoscope (25)/ endoscope polarizing cap. In this position the resistance beads (390) allow insertion and retraction movements of the rear view catheter (51) inside the hollow channel of the endoscope (25)/endoscope polarizing cap while preventing rotational movement of the rear view catheter (51) in relation to the endoscope by virtue of its resistance with the shaft of the rear view catheter (51). The resistance beads may alternatively be placed along the periphery of the rear view catheter.

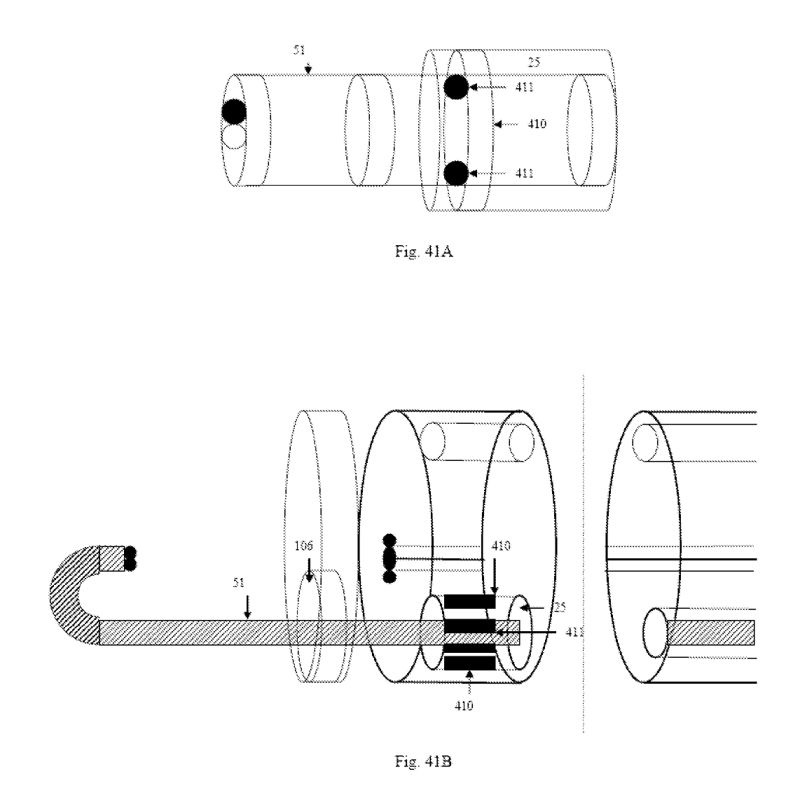

According to fourth embodiment, shown in FIG. 40, a locking means comprising of elevator knob (400) is provided in the hollow channel of the endoscope (25). The elevator knob (400) is operatively connected by pull wires (401) to an actuator (402) at the proximal end of the endoscope. Actuation of the pull wires (401) moves elevator knob (400) outwards and inwards into the hollow channel of the endoscope (25). The elevator knob (400) has a V or U shaped groove (not shown) of a size to accommodate the rear view catheter (51) along its groove. Operationally, once the rear view catheter (51) is pushed out of the tip of the endoscope, the elevator knob (400) is moved inwards into the hollow channel (25); at which time the groove on the elevator knob (400) securely engages the rear view catheter (51); thereby preventing its rotational movement with respect to the endoscope. At the end of the procedure, the elevator knob (400) is moved outwards thereby disengaging from the rear view catheter (51), following which the rear view catheter (51) is pulled out.

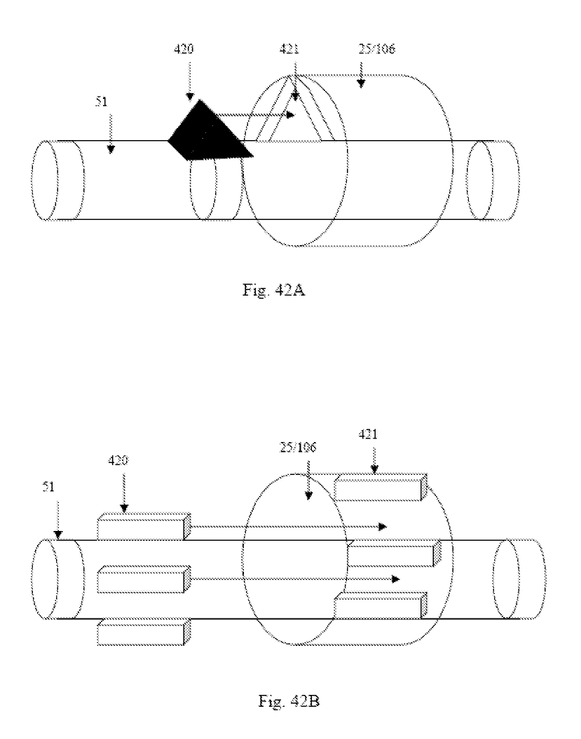

According to fifth embodiment, shown in FIGS. 41A & 41B, magnetic ring (410) is attached to the inner surface of the distal end of the hollow channel of the endoscope (25)/endoscope polarizing cap (106). Metal ring (411) is attached to the distal end of the rear view catheter (51); at a pre determined distance from its tip housing the rear image lens. Preferably, the metal ring (411) is disposed at a distance from the tip of the rear view catheter such that the rear view catheter is able to achieve satisfactory retro flexed position and the rear image lens is able to provide satisfactory rear view of a hollow organ upon exit from the distal tip of the endoscope. Operationally, as shown in FIG. 41B, when the rear view catheter (51) is far enough out of the endoscope tip; the metal ring (411) engages the magnet ring (410) disposed inside the hollow channel of the endoscope (25)/endoscope polarizing cap (106); thereby securing the position of the rear view catheter (51) and preventing rotational movement with respect to the shaft of the endoscope. The positions of the magnet rings (410) and metal rings (411) can be interchanged while achieving similar end results, and should not be considered limiting.

According to sixth embodiment, shown in FIGS. 42A & 42B, locking assembly is provided comprising of; 1) a locking contraption (420) attached to the distal part of the rear view catheter; and 2) mating contraption (421) corresponding to locking contraption (420) attached to the hollow channel of the distal end of endoscope (25) and/or endoscope polarizing cap (106) attached thereto. Operationally, upon exit from the hollow channel of the endoscope (25)/endoscope polarizing cap (106), the locking contraption (420) reversibly engages with the mating contraption (421); thereby preventing rotational movement of the rear view catheter (51). The engagement of the locking and mating contraptions (420, 421) preferably happens automatically upon exit of the rear view catheter (51) from endoscope/endoscope polarizing cap to a pre determined distance; or alternately user may be required to pull the rear view catheter (51) backwards upon exit from the endoscope/endoscope polarizing cap to enable engagement of the mating and locking contraptions (420, 421). At the end of the procedure; the locking contraption (420) is disengaged from the mating contraption (421) and the rear view catheter (51) is pulled out of the endoscope. The locking contraption (420) and corresponding mating contraption (421) may be of varied shapes; for example, rectangular bars, spherical beads etc. The locking contraption (420) is made of flexible material; preferably shape memory material, that when pushed thorough the hollow channel of the endoscope (25) contracts; and once outside of the distal end of the endoscope resumes original shape and locks with corresponding mating contraption (421). An example of this is shown in FIG. 42A, where the locking contraption (420) comprises of two ‘V’ shaped bars made of shape memory material and is disposed along opposite sides of the outer periphery of the distal end of the rear view catheter (51). Mating contraption (421) corresponding to the two ‘V’ shaped bars is provided in the hollow channel of the distal end of the endoscope (25)/endoscope polarizing cap (106). Upon exit from the hollow channel of the endoscope (25)/endoscope polarizing cap (106); the ‘V’ shaped locking contraption (420) resumes its ‘V’ shape; following which the rear view catheter (51) is gently pulled back to enable the ‘V’ shaped locking contraption (420) to engage with corresponding mating contraption (421), thus stabilizing the rear view catheter. According to another example shown in FIG. 42B, where the locking contraption (420) comprises of rectangular bars made of shape memory material and is disposed along opposite sides of the outer periphery of the distal end of the rear view catheter (51). Mating contraption (421) corresponding to the rectangular bars comprises of grooves between rectangular bars strategically placed along the circumference of the hollow channel of the distal end of the endoscope (25)/endoscope polarizing cap (106). Upon exit from the hollow channel of the endoscope (25)/endoscope polarizing cap (106); the rectangular bars (420) engage with corresponding mating contraption (421); thus stabilizing the rear view catheter (51) and prevent its rotational movement with respect to the shaft of the endoscope.

Any person/persons familiar with prior art will understand that modifications or changes to the present invention can be made without compromising its principles. Such modifications or changes should be considered inclusive and not limiting.

What is claimed is:

1. An endoscope system comprising of; 1) first endoscope assembly comprising of first image lens and first illumination source disposed in first direction; 2) second endoscope assembly comprising of second image lens disposed in second direction; 3) the first illumination source facing the second image lens; and 4) polarizing filter having means to allow selective passage of light disposed between first and second endoscope assemblies.

2. The endoscope system of claim 1; further comprising of second illumination source disposed on second endoscope assembly; and wherein first polarizing filter pair having identical orientation is disposed in front of first image lens and first illumination source; second polarizing filter pair having identical orientation is disposed in front of second image lens and second illumination source; second polarizing filter orientation at an angle to first polarizing filter.

3. The endoscope system of claim 1; further comprising of second illumination source disposed on second endoscope assembly; first polarizing filter pair having identical orientation is disposed in front of first image lens and second illumination source; second polarizing filter pair having identical orientation disposed in front of second image lens and first illumination source; second polarizing filter orientation at an angle to first polarizing filter.

4. The endoscope system of claim 1; further comprising; first polarizing filter disposed in front of first image lens and first illumination source; second polarizing filter is disposed in front of second endoscope assembly; second polarizing filter oriented at an angle to first polarizing filter.

5. The endoscope system of claim 1; wherein the polarizing filter is a linear filter or circular filter.

6. Endoscope system comprising of; 1) first endoscope assembly comprising of first image lens and first illumination source disposed in first direction; 2) second endoscope assembly comprising of second image lens and second illumination source disposed in second direction; 3) the first illumination source facing the second image lens; and second illumination source facing the first image lens; 4) light adjustment means for first and second illumination sources.

7. The endoscope system of claim 6; wherein light adjustment means comprises of light output adjustment system for illumination bulbs corresponding to light source.

8. The endoscope system of claim 6; wherein light adjustment means comprises of image light exposure adjustment system.

9. The endoscope system of claim 6; wherein light adjustment means comprises of light output having first wavelength from first illumination source and light output having second wavelength from second light source.

10. An endoscope system comprising of; 1) first endoscope assembly comprising of first image lens and first illumination source disposed in first direction; 2) second endoscope assembly comprising of second image lens and second illumination source disposed in second direction; 3) the first illumination source facing the second image lens; and second illumination source facing the first image lens; 4) first endoscope assembly independently movable from second endoscope assembly; 5) means for alternate activation of first and second illumination sources.

11. The endoscope system of claim 10; wherein activation means for first and second illumination sources comprises of alternate power supply to first and second illumination source respectively.

12. The endoscope system of claim 10; wherein alternate activation means for first and second illumination sources comprises of first and second shutter screens disposed in front of first and second illumination sources, and having means for alternate positioning of first and second shutter screens in front of first and second illumination sources.

13. The endoscope system of claim 12; wherein shutter screen comprised of circular sheet having partial opaque and partial transparent surface.

14. The endoscope system of claim 12; wherein shutter screen comprises of opaque sheet.

15. The endoscope system of claim 10; wherein illumination source comprises of illumination bulb disposed at light output port.

16. The endoscope system of claim 10; wherein illumination source comprises of remote illumination bulb and light guide extending from remote illumination bulb to illumination output port.

17. An endoscope system having; 1) insertion tube having first image lens and first illumination source disposed at distal end; 2) catheter assembly extendable from insertion tube having second image lens; 3) second illumination source disposed on insertion tube proximal to first illumination bulb; and 4) second illumination source disposed at and angle to the first illumination bulb.

18. The endoscope system of claim 17; wherein hollow channel extends from proximal to distal end of the insertion tube and catheter assembly retractably extends through hollow channel of the insertion tube.

19. The endoscope system of claim 17; wherein second illumination source is an illumination bulb disposed at light output port.

20. The endoscope system of claim 17; wherein second illumination source comprises of remote illumination bulb and light guide extending from remote illumination bulb to light output port disposed on distal end of insertion tube.
